(19) United States
(12) Patent Application Publication
(10) Pub. No.: US 2024/0131084 A1
(43) Pub. Date: Apr. 25, 2024

(54) LYOPHILIZED COMPOSITIONS COMPRISING FECAL MICROBE-BASED THERAPEUTIC AGENTS AND METHODS FOR MAKING AND USING SAME

(71) Applicant: Finch Therapeutics Holdings LLC, Boston, MA (US)

(72) Inventor: Bharat DIXIT, Acton, MA (US)

(73) Assignee: Finch Therapeutics Holdings LLC, Boston, MA (US)

(21) Appl. No.: 18/392,435

(22) Filed: Dec. 21, 2023

Related U.S. Application Data
(63) Continuation of application No. 16/616,846, filed on Nov. 25, 2019, now Pat. No. 11,890,306.
(60) Provisional application No. 62/511,776, filed on May 26, 2017.

(51) Int. Cl. A61K 35/74; A61K 9/19; A61K 47/18; A61K 47/22; A61K 47/26

(52) U.S. Cl. CPC A61K 35/74; A61K 9/19; A61K 47/18; A61K 47/22; A61K 47/26; A61K 9/0053

(57) ABSTRACT

This application provides compositions, e.g., formulations, used for gastric, gastrointestinal and/or colonic treatments, and methods for making, storing and using them, including formulations that effectively preserve microbial cell viability during freeze-thaw or freeze-drying. Compositions provided herein are useful for treating various diseases or conditions such as autism spectrum disordor, Crohn's Disease, ulcerative colitis, irritable bowel syndrome, and recurrent or primary C. difficile infection.

### CROSS-REFERENCE TO RELATED APPLICATION

This application claims priority to U.S. Provisional Application No. 62/511,776, filed May 26, 2017, which is incorporated by reference herein in its entirety.

### TECHNICAL FIELD

This disclosure generally relates to medicine and gastroenterology, pharmacology and microbiology. In one aspect, compositions provided herein are used for the stabilization, amelioration, treatment and/or prevention of constipation, for the treatment of abdominal pain, particularly non-specific abdominal pain, and diarrhea, including diarrhea caused by a drug side effect, a psychological condition, a disease or a condition such as autism, Crohn's Disease, a poison, a toxin or an infection, e.g., a toxin-mediated traveller's diarrhea, or C. difficile or the pseudo-membranous colitis associated with this infection. In one aspect, pharmaceuticals and products (articles) of manufacture provided herein are delivered to an individual, e.g., a human or an animal, in need thereof.

### BACKGROUND

Implantation or administration of human colonic microbiota into the bowel of a sick patient is often referenced as Fecal Microbiota Transplantation (FMT). It is a therapeutic process originally designed to treat Clostridium difficile infection (CDI) and others. It entails infusions through a colonoscope, an enema or via a nasojejunal tube of human microbiota either in the form of homogenized stool, extracts of homogenized stool, or cultured stool components such as Clostridia, to implant in the colon and so displace or eradicate the pathogenic Clostridium difficile; and it has a high success rate. In treating C. difficile infection, FMT is a highly efficacious treatment which carries well over a 90% cure rate with a single infusion and higher rate with multiple infusions. Hence, FMT can be life-saving given the current CDI mortality in the US of some 30,000 persons/year. Such a therapeutic process has also been used in treating other gut infective agents such as E. coli and Vancomycin resistant Enterococci (VRE).

There is growing demand for FMT primarily for the treatment of CDI. However, use of the therapy is restricted by the logistics of obtaining fresh FMT material from pre-screened donors in a timely fashion. Access to pre-prepared FMT material, stored frozen or lyophilized, would improve access to the therapy. Here, Applicant provides, among other uses, the production and use of lyophilized pharmaceutical compositions comprising a lyophilization formulation that uses a cryoprotectant, such as trehalose with or without a reducing agent such as cysteine, sodium ascorbate, or ascorbic acid to preserve microbial cell viability at an unexpectedly high efficiency.

### SUMMARY

In one aspect, the present disclosure provides a pharmaceutical composition comprising a lyophilized fecal microbe preparation comprising a lyophilization formulation comprising at least about 12.5% trehalose, wherein the lyophilization formulation is capable of providing a cell integrity maintenance ratio of at least about 20%. In another aspect, a pharmaceutical composition comprises a lyophilization formulation further comprising cysteine selected from the group consisting of D-cysteine and L-cysteine.

In another aspect, the present disclosure provides a lyophilized fecal microbe composition comprising a lyophilization formulation comprising trehalose and cysteine, wherein the lyophilization formulation is capable of maintaining at least about 30% cell viability immediately post lyophilization relative to a pre-lyophilization cell viability.

In one aspect, the present disclosure provides lyophilized fecal microbiota pharmaceutical composition comprising a lyophilization formulation comprising at least about 12.5% trehalose, wherein the lyophilization formulation is capable of providing a cell integrity maintenance ratio of at least 35%.

In one aspect, the present disclosure provides a method of preparing a lyophilized fecal microbe preparation, the method comprising:
a. obtaining a liquid fecal microbe preparation in a lyophilization formulation comprising between 12.5% and 17.5% trehalose; and
b. freeze-drying the liquid fecal microbe preparation, wherein the lyophilization formulation is capable of providing a cell integrity maintenance ratio of at least about 30%.

In another aspect, the present disclosure provides a frozen fecal microbe composition comprising trehalose, wherein the frozen fecal microbe composition is capable of being thawed at least once without loss of microbial cell membrane integrity of more than 50%. In another aspect, a frozen fecal microbe composition further comprises cysteine selected from the group consisting of D-cysteine and L-cysteine.

In another aspect, this disclosure provides a method for treating a disorder or condition in a subject in need thereof, comprising administering to the subject a therapeutically effective amount of a pharmaceutical composition or a previously frozen fecal microbe composition disclosed herein, wherein the disorder or condition is selected from the group consisting of Acne, AIDS Enteropathy, AIDS-related Gastroenteritis, Alopecia Totalis, Alzheimers Disease, Amyloidosis, Amyotrophic Lateral Sclerosis, Ankylosing Spondylitis, Anorexia, Antibiotic Associated Colitis, Asbergers Syndrome, Attention Deficit Disorder (ADD), Attention Deficit Hyperactivity Disorder (ADHD), Autism Spectrum Disorder (ASD), Behcet's Syndrome, Chronic Clostridium difficile Infection (CDI), Chronic constipation, Chronic Depression, Chronic Fatigue Syndrome (CFS), Chronic Idiopathic Pseudo Obstructive Syndrome, Chronic Inflammation Demyelinating Polyneuropathy, Chronic Nausea, Chronic Urticaria, Coeliac Disease, Collagenous Colitis, Colonic Polyps, Constipation Predominant FBD, Crohn's Disease, Cryptogenic Cirrhosis, Cyclic Vomiting, Dermatitis Herpetiformis, Diabetes, Familial Mediterranean Fever, Fatty Liver, Functional Bowel Disease (FBD), Gastro-oesophageal Reflux, Gillian-Barre Syndrome, Glomerulonephritis, Haemolytic Uraemic Syndrome, Halitosis, IBS constipation-predominant, IBS diarrhea/constipation alternating, IBS diarrhea-predominant, IBS pain-predominant, Idiopathic Thrombocytopenic Purpura (ITP), Idiopathic/Simple Constipation, Indeterminate Colitis, Inflammatory Bowel Disease (IBD), Irritable bowel syndrome (IBS), Juvenile Diabetes Mellitus, Lyme Disease, Manic Depressive Illness, Metabolic Syndrome, Microscopic Colitis, Migraine, Mixed Cryoglobulinaemia, Mucous Colitis, Multiple Sclerosis, Myasthenia Gravis, NASH (Nonalcoholic Steatohepatitis), Non-Rheumatoid Arthritis, Non-Rheumatoid Factor Positive Arthritis, Non-ulcer Dyspepsia, Norwalk Viral Gastroenteritis, Obesity, Obsessive Compulsive Disorder, Pain Predominant FBD, Parkinson's Disease, Polyarteritis, Polyposis Coli, Primary Biliary Cirrhosis, Primary Clostridium difficile Infection (CDI), Primary Sclerosing Cholangitis (PSC), Pseudomembranous Colitis, Psychotic Disorders, Reiter's Syndrome, Relapsing Diverticulitis, Rett Syndrome, Rheumatoid Arthritis, Rosacea, Rotavirus Gastroenteritis, Sacroiliitis, Schizophrenia, Scleroderma, Sjogren's Syndome, Small Bowel Bacterial Overgrowth, Sudden Infant Death Syndrome (SIDS), Systemic Lupus Erythematosus, Ulcerative Colitis, Upper Abdominal FBD, Vasculitic Disorders, Viral Gastroenteritis, pre-diabetic syndrome, type I diabetes, type II diabetes, depression, schizophrenia, and a mood disorder.

In another aspect, this disclosure provides a method for treating a disorder or condition in a subject in need thereof, comprising administering to the subject a therapeutically effective amount of a pharmaceutical composition or a previously frozen fecal microbe composition disclosed herein, wherein the disorder or condition is selected from the group consisting of recurrent or primary C. diff infection, autism spectrum disorder (ASD), ulcerative colitis, Crohn's disease, and irritable bowel syndrome.

### BRIEF DESCRIPTION OF THE DRAWINGS

FIG. 1. shows a flowchart illustrating an exemplary process for manufacturing a composition described herein.

### DETAILED DESCRIPTION

Unless defined otherwise herein, terms are to be understood according to conventional usage by those of ordinary skill in the relevant art.

As used herein, “lyophilization” or “freeze drying” refers to the process of drying a material by first freezing it and then encouraging the ice within it to sublimate in a vacuum environment.

As used herein, a “cell integrity maintenance ratio” is defined as the percentage of microbial cells maintaining integral cell membrane post-lyophilization relative to pre-lyophilization. As used herein, “microbial cell membrane integrity” refers to the actual percent of microbial cells that possess integral cell membrane in a population of microbes. As used herein, a “post-lyophilization cell membrane integrity percentage” refers to the percentage of microbial cells with cell membrane being intact in a lyophilized composition. The “cell integrity maintenance ratio” is based on a comparison between post-lyophilization and pre-lyophilization and thus reflects changes in cell integrity after a lyophilization process. The “microbial cell membrane integrity” and “post-lyophilization cell membrane integrity percentage” reflect the actual percentage of cells that contain integral cell membrane in a composition. Membrane integrity can be measured using a variety of methods including flow cytometry-based method and fluorescence microscope-based method. Cells can be stained with dyes that specifically recognize cells with intact, integral, or compromised membranes. In some instances multiple dyes can be used simultaneously to detect live and dead cells in a population of cells. For example, one aliquot of cells is stained with SYTO™ BC dye (Invitrogen, CA) to stain cells and Prolong Anti-Fade Reagent (Invitrogen, CA) to minimize photo-bleaching of dye. A second aliquot is stained with another dye, Propidium Iodide (Invitrogen, CA). Whereas the SYTO BC dye penetrates all cells which stain green, the Propidium Iodide (PI) dye only penetrates cells with a loss of membrane integrity (i.e. injured or dead cells), and the PI stained cells appear red when visualized under the epifluorescence microscope using the specific red filter. Details of an exemplary cell staining protocol can be found in Example 2.

As used herein, “viable” means possessing the ability to multiply. Here, the viability of bacterial populations is monitored as a function of the membrane integrity of the cell. Cells with a compromised membrane are considered to be dead or dying (which include injured cells or cells with compromised membrane that are live but unable to divide), whereas cells with an intact membrane are considered live.

As used herein, unless indicated otherwise, percent concentration refers to weight-to-volume percent (% w/v). For example, 15% trehalose refers to a solution which contains 15 g of trehalose per 100 mL of solution.

As used herein, a “cryoprotectant” refers to a substance that is added to a formulation in order to protect an active ingredient during freezing.

As used herein, a “reducing agent” refers to a substance that is added to a formulation in order to bring about reduction by being oxidized. In an aspect of the present disclosure, a reducing agent is selected from the group comprising cysteine, ascorbic acid, sodium ascorbate, thioglycolic acid, sodium sulfite, sodium bisulfite, sodium metabisulfite, potassium metabisulfite, Glutathione, Methionine, thioglycerol, and alpha tocopherol.

As used herein, a “lyoprotectant” refers to a substance that is added to a formulation in order to protect an active ingredient during the drying stage of a lyophilization (also known as freeze-drying) process.

As used herein, a “microbiota” and “flora” refer to a community of microbes that live in or on a subject's body, both sustainably and transiently, including eukaryotes, archaea, bacteria, and viruses (including bacterial viruses (i.e., phage)). A “fecal microbiota” or “fecal microbiota preparation” refers to a community of microbes present in a subject's feces. A non-selected fecal microbiota refers to a community or mixture of fecal microbes derived from a donor's fecal sample without selection and substantially resembling microbial constituents and population structure found in such fecal sample.

As used herein, “colony forming units” (cfu) refers to an estimate of the number of viable microorganism cells in a given sample.

As used herein, “isolated” or “purified” refers to a bacterium or other entity or substance that has been (1) separated from at least some of the components with which it was associated when initially produced (whether in nature or in an experimental setting), and/or (2) produced, prepared, purified, and/or manufactured by the hand of man. Isolated or purified bacteria can be separated from at least about 10%, about 20%, about 30%, about 40%, about 50%, about 60%, about 70%, about 80%, about 90%, about 92%, about 94%, about 96%, about 98%, about 99% or more of the other components with which they were initially associated.

As used herein, the terms “pathogen” and “pathogenic” in reference to a bacterium or any other organism or entity includes any such organism or entity that is capable of causing or affecting a disease, disorder or condition of a host organism containing the organism or entity.

As used herein, “spore” or a population of “spores” includes bacteria (or other single-celled organisms) that are generally viable, more resistant to environmental influences such as heat and bacteriocidal agents than vegetative forms of the same bacteria, and typically capable of germination and out-growth. “Spore-formers” or bacteria “capable of forming spores” are those bacteria containing the genes and other necessary abilities to produce spores under suitable environmental conditions.

As used herein, “subject” refers to any animal subject including humans, laboratory animals (e.g., primates, rats, mice), livestock (e.g., cows, sheep, goats, pigs, turkeys, chickens), and household pets (e.g., dogs, cats, rodents, etc.). The subject or patient may be healthy, or may be suffering from an infection due to a gastrointestinal pathogen or may be at risk of developing or transmitting to others an infection due to a gastrointestinal pathogen.

As used herein, “Shannon Diversity Index” refers to a diversity index that accounts for abundance and evenness of species present in a given community using the formula H=−Σᵢ₌₁^{R}pᵢ ln pᵢ, where H is Shannon Diversity Index, R is the total number of species in the community, and p; is the proportion of R made up of the ith species. Higher values indicate diverse and equally distributed communities, and a value of 0 indicates only one species is present in a given community. For further reference, see Shannon and Weaver, (1949) The mathematical theory of communication. The University of Illinois Press, Urbana. 117 pp.

As used herein, “antibiotic” refers to a substance that is used to treat and/or prevent bacterial infection by killing bacteria, inhibiting the growth of bacteria, or reducing the viability of bacteria.

As used herein, the term “treating” refers to (i) completely or partially inhibiting a disease, disorder or condition, for example, arresting its development; (ii) completely or partially relieving a disease, disorder or condition, for example, causing regression of the disease, disorder and/or condition; or (iii) completely or partially preventing a disease, disorder or condition from occurring in a patient that may be predisposed to the disease, disorder and/or condition, but has not yet been diagnosed as having it. Similarly, “treatment” refers to both therapeutic treatment and prophylactic or preventative measures.

As used herein, “therapeutically effective amount” or “pharmaceutically active dose” refers to an amount of a composition which is effective in treating the named disease, disorder or condition.

As used herein, the term “substantially”, when used to modify a quality, generally allows certain degree of variation without that quality being lost. For example, in certain aspects such degree of variation can be less than 0.1%, about 0.1%, about 0.2%, about 0.3%, about 0.4%, about 0.5%, about 0.6%, about 0.7%, about 0.8%, about 0.9%, about 1%, between 1-2%, between 2-3%, between 3-4%, between 4-5%, or greater than 5%.

In one aspect, the present disclosure provides a pharmaceutical composition comprising a lyophilized fecal microbe preparation comprising a lyophilization formulation comprising at least about 12.5% trehalose, wherein the lyophilization formulation is capable of providing a cell integrity maintenance ratio of at least about 30%.

In one aspect, a lyophilization formulation comprises at least about 5%, at least about 7.5%, at least about 10%, at least about 12.5%, at least about 13%, at least about 13.5%, at least about 14%, at least about 14.5%, at least about 15%, at least about 15.5%, at least about 16%, at least about 16.5%, at least about 17%, at least about 17.5%, at least about 18%, at least about 18.5%, at least about 19%, at least about 19.5%, at least about 20%, at least about 22.5%, at least about 25%, at least about 27.5%, at least about 30%, at least about 32.5%, at least about 35%, at least about 37.5%, at least about 40%, at least about 42.5%, at least about 45%, at least about 47.5%, at least about 50%, at least about 52.5%, at least about 55%, at least about 57.5%, or at least about 60% trehalose.

In another aspect, a lyophilization formulation comprises between about 12.5% and about 60%, between about 13% and about 60%, between about 13.5% and about 60%, between about 14% and about 60%, between about 14.5% and about 60%, between about 15% and about 60%, between about 15.5% and about 60%, between about 16% and about 60%, between about 16.5% and about 60%, between about 17% and about 60%, between about 17.5% and about 60%, between about 18% and about 60%, between about 18.5% and about 60%, between about 19% and about 60%, between about 19.5% and about 60%, between about 20% and about 60%, between about 22.5% and about 60%, between about 25% and about 60%, between about 27.5% and about 60%, between about 30% and about 60%, between about 32.5% and about 60%, between about 35% and about 60%, or between about 37.5% and about 60% trehalose.

In one aspect, a lyophilization formulation comprises between about 12.5% and about 40%, between about 13% and about 40%, between about 13.5% and about 40%, between about 14% and about 40%, between about 14.5% and about 40%, between about 15% and about 40%, between about 15.5% and about 40%, between about 16% and about 40%, between about 16.5% and about 40%, between about 17% and about 40%, between about 17.5% and about 40%, between about 18% and about 40%, between about 18.5% and about 40%, between about 19% and about 40%, between about 19.5% and about 40%, between about 20% and about 40%, between about 22.5% and about 40%, between about 25% and about 40%, between about 27.5% and about 40%, between about 30% and about 40%, between about 32.5% and about 40%, between about 35% and about 40%, or between about 37.5% and about 40% trehalose.

In another aspect, a lyophilization formulation comprises between about 12.5% and about 30%, between about 13% and about 30%, between about 13.5% and about 30%, between about 14% and about 30%, between about 14.5% and about 30%, between about 15% and about 30%, between about 15.5% and about 30%, between about 16% and about 30%, between about 16.5% and about 30%, between about 17% and about 30%, between about 17.5% and about 30%, between about 18% and about 30%, between about 18.5% and about 30%, between about 19% and about 30%, between about 19.5% and about 30%, between about 20% and about 30%, between about 22.5% and about 30%, between about 25% and about 30%, or between about 27.5% and about 30% trehalose.

In one aspect, a lyophilization formulation comprises between about 12.5% and about 25%, between about 13% and about 25%, between about 13.5% and about 25%, between about 14% and about 25%, between about 14.5% and about 25%, between about 15% and about 25%, between about 15.5% and about 25%, between about 16% and about 25%, between about 16.5% and about 25%, between about 17% and about 25%, between about 17.5% and about 25%, between about 18% and about 25%, between about 18.5% and about 25%, between about 19% and about 25%, between about 19.5% and about 25%, between about 20% and about 25%, or between about 22.5% and about 25% trehalose.

In another aspect, a lyophilization formulation comprises between about 12.5% and about 20%, between about 13% and about 20%, between about 13.5% and about 20%, between about 14% and about 20%, between about 14.5% and about 20%, between about 15% and about 20%, between about 15.5% and about 20%, between about 16% and about 20%, between about 16.5% and about 20%, between about 17% and about 20%, between about 17.5% and about 20%, between about 18% and about 20%, between about 18.5% and about 20%, between about 19% and about 20%, or between about 19.5% and about 20% trehalose.

In one aspect, a lyophilization formulation comprises between about 12.5% and about 17.5%, between about 13% and about 17.5%, between about 13.5% and about 17.5%, between about 14% and about 17.5%, between about 14.5% and about 17.5%, between about 15% and about 17.5%, between about 15.5% and about 17.5%, between about 16% and about 17.5%, between about 16.5% and about 17.5%, or between about 17% and about 17.5% trehalose.

In another aspect, a lyophilization formulation comprises between about 12.5% and about 17.5%, between about 13% and about 17%, between about 13.5% and about 16.5%, between about 14% and about 16%, or between about 14.5% and about 15.5% trehalose.

In one aspect, a lyophilization formulation comprises about 15% trehalose. In another aspect, a lyophilization formulation comprises 15% trehalose.

In one aspect, a lyophilization formulation is capable of providing a cell integrity maintenance ratio of at least about 25%, at least about 27.5%, at least about 30%, at least about 32.5%, at least about 35%, at least about 37.5%, at least about 40%, at least about 42.5%, at least about 45%, at least about 47.5%, at least about 50%, at least about 52.5%, at least about 55%, at least about 57.5%, at least about 60%, at least about 62.5%, at least about 65%, at least about 67.5%, at least about 70%, at least about 72.5%, at least about 75%, at least about 77.5%, at least about 80%, at least about 82.5%, at least about 85%, at least about 87.5%, at least about 90%, at least about 92.5%, or at least about 95%.

In another aspect, a lyophilization formulation is capable of providing a cell integrity maintenance ratio of between about 32.5% and about 90%, between about 35% and about 90%, between about 37.5% and about 90%, between about 40% and about 90%, between about 42.5% and about 90%, between about 45% and about 90%, between about 47.5% and about 90%, between about 50% and about 90%, between about 52.5% and about 90%, between about 55% and about 90%, between about 57.5% and about 90%, between about 60% and about 90%, between about 62.5% and about 90%, between about 65% and about 90%, between about 67.5% and about 90%, between about 70% and about 90%, between about 72.5% and about 90%, between about 75% and about 90%, between about 77.5% and about 90%, between about 80% and about 90%, between about 82.5% and about 90%, between about 85% and about 90%, or between about 87.5% and about 90%.

In one aspect, a lyophilization formulation is capable of providing a cell integrity maintenance ratio of between about 32.5% and about 80%, between about 35% and about 80%, between about 37.5% and about 80%, between about 40% and about 80%, between about 42.5% and about 80%, between about 45% and about 80%, between about 47.5% and about 80%, between about 50% and about 80%, between about 52.5% and about 80%, between about 55% and about 80%, between about 57.5% and about 80%, between about 60% and about 80%, between about 62.5% and about 80%, between about 65% and about 80%, between about 67.5% and about 80%, between about 70% and about 80%, between about 72.5% and about 80%, between about 75% and about 80%, or between about 77.5% and about 80%.

In another aspect, a lyophilization formulation is capable of providing a cell integrity maintenance ratio of between about 32.5% and about 70%, between about 35% and about 70%, between about 37.5% and about 70%, between about 40% and about 70%, between about 42.5% and about 70%, between about 45% and about 70%, between about 47.5% and about 70%, between about 50% and about 70%, between about 52.5% and about 70%, between about 55% and about 70%, between about 57.5% and about 70%, between about 60% and about 70%, between about 62.5% and about 70%, between about 65% and about 70%, or between about 67.5% and about 70%.

In one aspect, a lyophilization formulation is capable of providing a cell integrity maintenance ratio of between about 32.5% and about 60%, between about 35% and about 60%, between about 37.5% and about 60%, between about 40% and about 60%, between about 42.5% and about 60%, between about 45% and about 60%, between about 47.5% and about 60%, between about 50% and about 60%, between about 52.5% and about 60%, between about 55% and about 60%, or between about 57.5% and about 60%.

In another aspect, a lyophilization formulation is capable of providing a cell integrity maintenance ratio of between about 32.5% and about 50%, between about 35% and about 50%, between about 37.5% and about 50%, between about 40% and about 50%, between about 42.5% and about 50%, between about 45% and about 50%, or between about 47.5% and about 50%.

In one aspect, a lyophilization formulation is capable of providing a cell integrity maintenance ratio of between about 32.5% and about 80%, between about 35% and about 77.5%, between about 37.5% and about 75%, between about 40% and about 67.5%, between about 42.5% and about 65%, between about 45% and about 62.5%, between about 47.5% and about 60%, between about 50% and about 57.5%, or between about 52.5% and about 55%.

In another aspect, a lyophilization formulation is capable of providing a cell integrity maintenance ratio of between about 32.5% and about 90%, between about 35% and about 87.5%, between about 37.5% and about 85%, between about 40% and about 82.5%, between about 42.5% and about 77.5%, between about 45% and about 75%, between about 47.5% and about 72.5%, between about 50% and about 70%, between about 52.5% and about 67.5%, between about 55% and about 65%, or between about 57.5% and about 62.5%.

In one aspect, a lyophilization formulation is capable of providing a post-lyophilization cell membrane integrity percentage of at least about 20%, at least about 22.5%, at least about 25%, at least about 27.5%, at least about 30%, at least about 32.5%, at least about 35%, at least about 37.5%, at least about 40%, at least about 42.5%, at least about 45%, at least about 47.5%, at least about 50%, at least about 52.5%, at least about 55%, at least about 57.5%, at least about 60%, at least about 62.5%, at least about 65%, at least about 67.5%, at least about 70%, at least about 72.5%, at least about 75%, at least about 77.5%, at least about 80%.

In one aspect, a lyophilization formulation is capable of providing a post-lyophilization cell membrane integrity percentage of between about 20% and about 80%, between about 22.5% and about 80%, between about 25% and about 80%, between about 27.5% and about 80%, between about 30% and about 80%, between about 32.5% and about 80%, between about 35% and about 80%, between about 37.5% and about 80%, between about 40% and about 80%, between about 42.5% and about 80%, between about 45% and about 80%, between about 47.5% and about 80%, between about 50% and about 80%, between about 52.5% and about 80%, between about 55% and about 80%, between about 57.5% and about 80%, between about 60% and about 80%, between about 62.5% and about 80%, between about 65% and about 80%, between about 67.5% and about 80%, between about 70% and about 80%, between about 72.5% and about 80%, between about 75% and about 80%, or between about 77.5% and about 80%.

In one aspect, a lyophilization formulation is capable of providing a post-lyophilization cell membrane integrity percentage of between about 20% and about 70%, between about 22.5% and about 70%, between about 25% and about 70%, between about 27.5% and about 70%, between about 30% and about 70%, between about 32.5% and about 70%, between about 35% and about 70%, between about 37.5% and about 70%, between about 40% and about 70%, between about 42.5% and about 70%, between about 45% and about 70%, between about 47.5% and about 70%, between about 50% and about 70%, between about 52.5% and about 70%, between about 55% and about 70%, between about 57.5% and about 70%, between about 60% and about 70%, between about 62.5% and about 70%, between about 65% and about 70%, or between about 67.5% and about 70%.

In one aspect, a lyophilization formulation is capable of providing a post-lyophilization cell membrane integrity percentage of between about 20% and about 60%, between about 22.5% and about 60%, between about 25% and about 60%, between about 27.5% and about 60%, between about 30% and about 60%, between about 32.5% and about 60%, between about 35% and about 60%, between about 37.5% and about 60%, between about 40% and about 60%, between about 42.5% and about 60%, between about 45% and about 60%, between about 47.5% and about 60%, between about 50% and about 60%, between about 52.5% and about 60%, between about 55% and about 60%, or between about 57.5% and about 60%.

In one aspect, a lyophilization formulation is capable of providing a post-lyophilization cell membrane integrity percentage of between about 20% and about 50%, between about 22.5% and about 50%, between about 25% and about 50%, between about 27.5% and about 50%, between about 30% and about 50%, between about 32.5% and about 50%, between about 35% and about 50%, between about 37.5% and about 50%, between about 40% and about 50%, between about 42.5% and about 50%, between about 45% and about 50%, or between about 47.5% and about 50%.

In one aspect, a lyophilization formulation is capable of providing a post-lyophilization cell membrane integrity percentage of between about 20% and about 40%, between about 22.5% and about 40%, between about 25% and about 40%, between about 27.5% and about 40%, between about 30% and about 40%, between about 32.5% and about 40%, between about 35% and about 40%, or between about 37.5% and about 40%.

In one aspect, a lyophilization formulation is capable of providing a post-lyophilization cell membrane integrity percentage of between about 32.5% and about 80%, between about 35% and about 77.5%, between about 37.5% and about 75%, between about 40% and about 67.5%, between about 42.5% and about 65%, between about 45% and about 62.5%, between about 47.5% and about 60%, between about 50% and about 57.5%, or between about 52.5% and about 55%.

In another aspect, a lyophilization formulation is capable of providing a post-lyophilization cell membrane integrity percentage of between about 32.5% and about 90%, between about 35% and about 87.5%, between about 37.5% and about 85%, between about 40% and about 82.5%, between about 42.5% and about 77.5%, between about 45% and about 75%, between about 47.5% and about 72.5%, between about 50% and about 70%, between about 52.5% and about 67.5%, between about 55% and about 65%, or between about 57.5% and about 62.5%.

In one aspect, a lyophilization formulation is capable of providing a post-lyophilization cell membrane integrity percentage of between about 25% and about 55%, between about 27.5% and about 52.5%, between about 30% and about 50%, between about 32.5% and about 47.5%, between about 35% and about 45%, or between about 37.5% and about 42.5%.

In one aspect, a pharmaceutical composition comprises a lyophilization formulation further comprising cysteine selected from the group consisting of D-cysteine and L-cysteine. In another aspect, cysteine is at a concentration of at least about 0.025%. In one aspect, cysteine is at a concentration of about 0.025%. In another aspect, cysteine is at a concentration of 0.025%. In another aspect, another reducing agent other than cysteine is used in lieu of, or in combination with cysteine. In an aspect, another reducing agent is selected from the group comprising ascorbic acid, sodium ascorbate, thioglycolic acid, sodium sulfite, sodium bisulfite, sodium metabisulfite, potassium metabisulfite, Glutathione, Methionine, thioglycerol, and alpha tocopherol. Any cysteine concentration mentioned in this disclosure is equally applicable to any of the foregoing reducing agents. For simplicity, concentration is not repeated. However, in all instances where a cysteine concentration or concentration range is mentioned, any one of the foregoing group of reducing agents is deemed also described with the same concentration or concentration ranges.

In one aspect, cysteine is at a concentration of at least about 0.005%, at least about 0.01%, at least about 0.015%, at least about 0.02%, at least about 0.025%, at least about 0.03%, at least about 0.035%, at least about 0.04%, at least about 0.045%, at least about 0.05%, at least about 0.055%, at least about 0.06%, at least about 0.065%, at least about 0.07%, at least about 0.075%, at least about 0.08%, at least about 0.085%, at least about 0.09%, at least about 0.095%, at least about 0.1%, at least about 0.12%, at least about 0.14%, at least about 0.16%, at least about 0.18%, at least about 0.2%, at least about 0.25%, at least about 0.3%, at least about 0.4%, at least about 0.5%, at least about 0.6%, at least about 0.7%, at least about 0.8%, at least about 0.9%, at least about 1%, at least about 2%, at least about 4%, at least about 6%, at least about 8%, at least about 10%, at least about 12%, at least about 14%, at least about 16%, at least about 18%, at least about 20%, at least about 22%, at least about 24%, or at least about 26%.

In another aspect, cysteine is at a concentration of between about 0.001% and about 0.005%, between about 0.005% and about 0.1%, between about 0.01% and about 0.1%, between about 0.015% and about 0.1%, between about 0.02% and about 0.1%, between about 0.025% and about 0.1%, between about 0.03% and about 0.1%, between about 0.035% and about 0.1%, between about 0.04% and about 0.1%, between about 0.045% and about 0.1%, between about 0.05% and about 0.1%, between about 0.055% and about 0.1%, between about 0.06% and about 0.1%, between about 0.065% and about 0.1%, between about 0.07% and about 0.1%, between about 0.075% and about 0.1%, between about 0.08% and about 0.1%, between about 0.085% and about 0.1%, between about 0.09% and about 0.1%, between about 0.095% and about 0.1%, between about 0.1% and about 0.3%, between about 0.3% and about 0.5%, between about 0.5% and about 0.7%, between about 0.7% and about 0.9%, between about 0.9% and about 1.0%, between about 0.1% and about 1.0%, between about 0.5% and about 1.0%.

In one aspect, cysteine is at a concentration of between about 0.005% and about 0.08%, between about 0.01% and about 0.08%, between about 0.015% and about 0.08%, between about 0.02% and about 0.08%, between about 0.025% and about 0.08%, between about 0.03% and about 0.08%, between about 0.035% and about 0.08%, between about 0.04% and about 0.08%, between about 0.045% and about 0.08%, between about 0.05% and about 0.08%, between about 0.055% and about 0.08%, between about 0.06% and about 0.08%, between about 0.065% and about 0.08%, between about 0.07% and about 0.08%, or between about 0.075% and about 0.08%.

In another aspect, cysteine is at a concentration of between about 0.005% and about 0.06%, between about 0.01% and about 0.06%, between about 0.015% and about 0.06%, between about 0.02% and about 0.06%, between about 0.025% and about 0.06%, between about 0.03% and about 0.06%, between about 0.035% and about 0.06%, between about 0.04% and about 0.06%, between about 0.045% and about 0.06%, between about 0.05% and about 0.06%, or between about 0.055% and about 0.06%.

In one aspect, cysteine is at a concentration of between about 0.005% and about 0.04%, between about 0.01% and about 0.04%, between about 0.015% and about 0.04%, between about 0.02% and about 0.04%, between about 0.025% and about 0.04%, between about 0.03% and about 0.04%, or between about 0.035% and about 0.04%.

In another aspect, cysteine is at a concentration of between about 0.005% and about 0.06%, between about 0.01% and about 0.05%, between about 0.015% and about 0.04%, or between about 0.02% and about 0.03%.

In another aspect, the present disclosure provides a lyophilized fecal microbe composition comprising a lyophilization formulation comprising trehalose and cysteine, wherein the lyophilization formulation is capable of maintaining at least about 30% cell viability immediately post lyophilization relative to a pre-lyophilization cell viability. In one aspect, a lyophilization formulation comprises at least 15% trehalose and at least 0.025% L-cysteine.

In one aspect, the present disclosure provides lyophilized fecal microbiota pharmaceutical composition comprising a lyophilization formulation comprising at least about 12.5% trehalose, wherein the lyophilization formulation is capable of providing a cell integrity maintenance ratio of at least 35%.

In one aspect, the present disclosure provides a method of preparing a lyophilized fecal microbe preparation, the method comprising:
a. obtaining a liquid fecal microbe preparation in a lyophilization formulation comprising between 12.5% and 17.5% trehalose; and
b. freeze-drying the liquid fecal microbe preparation, wherein the lyophilization formulation is capable of providing a cell integrity maintenance ratio of at least about 30%.

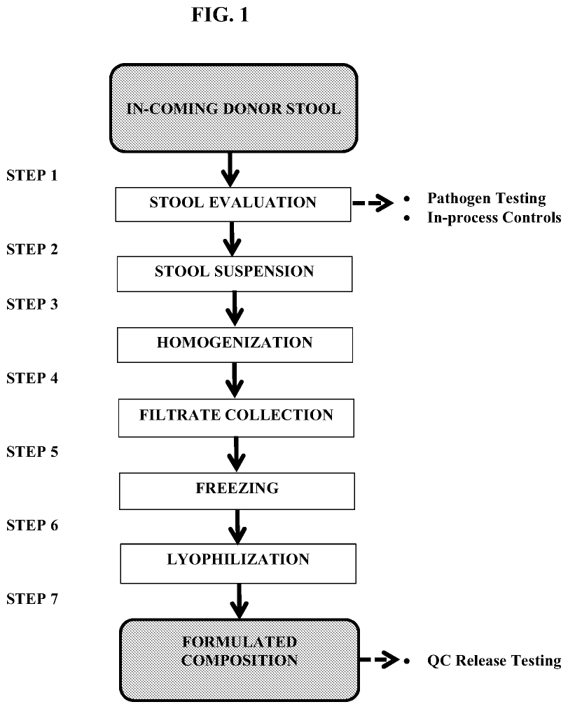

In another aspect, a method further comprises producing a liquid fecal microbe preparation from a stool in the absence of culturing of fecal microbes prior to mixing the stool with a homogenization buffer (see FIG. 1). For example, fresh feces can be directly homogenized in a buffer without culturing. In an aspect, unprocessed stool is directly mixed with a formulation described here (e.g., comprising trehalose, cysteine, or both) without culturing the fecal microbes.

FIG. 1 illustrates an example embodiment of a process for manufacturing a composition as described herein. At Step 1, fresh donor stool is evaluated for the presence of pathogens and the general health of the donor. At Step 2, the stool is combined with a liquid composition (e.g. homogenization buffer comprising saline, trehalose and reducing agent such as cysteine) to facilitate homogenization. At Step 3, the stool is homogenized in the liquid composition to produce a stool liquid suspension. At Step 4, the stool liquid suspension is filtered and the filtrate collected. At Step 5, the filtrate is frozen. At Step 6, the frozen filtrate is lyophilized to give rise a formulated composition as described herein. Such final composition can then be tested for safety and efficacy and administered to a subject in need thereof. In addition, in certain embodiments, the filtrate collected at Step 4 can be directly administered to a subject, without freezing or lyophilization.

In another aspect, the present disclosure provides a frozen fecal microbe composition comprising trehalose, wherein the frozen fecal microbe composition is capable of being thawed at least once without loss of microbial cell membrane integrity of more than 50%. In one aspect, a frozen fecal microbe composition is capable of being thawed at least 2, 3, 4, 5, or 6 times without loss of microbial cell membrane integrity of more than 50%. In another aspect, a frozen fecal microbe is capable of being thawed at least once, 2, 3, 4, 5, or 6 times without loss of microbial cell membrane integrity of more than 40%, more than 35%, more than 30%, more than 25%, more than 20%, more than 15%, more than 10%, or more than 5%. In another aspect, a frozen fecal microbe composition further comprises cysteine selected from the group consisting of D-cysteine and L-cysteine.

In another aspect, this disclosure provides a method for treating a disorder or condition in a subject in need thereof, comprising administering to the subject a therapeutically effective amount of a pharmaceutical composition or a previously frozen fecal microbe composition disclosed herein, wherein the disorder or condition is selected from the group consisting of Acne, AIDS Enteropathy, AIDS-related Gastroenteritis, Alopecia Totalis, Alzheimers Disease, Amyloidosis, Amyotrophic Lateral Sclerosis, Ankylosing Spondylitis, Anorexia, Antibiotic Associated Colitis, Asbergers Syndrome, Attention Deficit Disorder (ADD), Attention Deficit Hyperactivity Disorder (ADHD), Autism Spectrum Disorder (ASD), Behcet's Syndrome, Chronic Clostridium difficile Infection (CDI), Chronic constipation, Chronic Depression, Chronic Fatigue Syndrome (CFS), Chronic Idiopathic Pseudo Obstructive Syndrome, Chronic Inflammation Demyelinating Polyneuropathy, Chronic Nausea, Chronic Urticaria, Coeliac Disease, Collagenous Colitis, Colonic Polyps, Constipation Predominant FBD, Crohn's Disease, Cryptogenic Cirrhosis, Cyclic Vomiting, Dermatitis Herpetiformis, Diabetes, Familial Mediterranean Fever, Fatty Liver, Functional Bowel Disease (FBD), Gastro-oesophageal Reflux, Gillian-Barre Syndrome, Glomerulonephritis, Haemolytic Uraemic Syndrome, Halitosis, IBS constipation-predominant, IBS diarrhea/constipation alternating, IBS diarrhea-predominant, IBS pain-predominant, Idiopathic Thrombocytopenic Purpura (ITP), Idiopathic/Simple Constipation, Indeterminate Colitis, Inflammatory Bowel Disease (IBD), Irritable bowel syndrome (IBS), Juvenile Diabetes Mellitus, Lyme Disease, Manic Depressive Illness, Metabolic Syndrome, Microscopic Colitis, Migraine, Mixed Cryoglobulinaemia, Mucous Colitis, Multiple Sclerosis, Myasthenia Gravis, NASH (Nonalcoholic Steatohepatitis), Non-Rheumatoid Arthritis, Non-Rheumatoid Factor Positive Arthritis, Non-ulcer Dyspepsia, Norwalk Viral Gastroenteritis, Obesity, Obsessive Compulsive Disorder, Pain Predominant FBD, Parkinson's Disease, Polyarteritis, Polyposis Coli, Primary Biliary Cirrhosis, Primary Clostridium difficile Infection (CDI), Primary Sclerosing Cholangitis (PSC), Pseudomembranous Colitis, Psychotic Disorders, Reiter's Syndrome, Relapsing Diverticulitis, Rett Syndrome, Rheumatoid Arthritis, Rosacea, Rotavirus Gastroenteritis, Sacroiliitis, Schizophrenia, Scleroderma, Sjogren's Syndome, Small Bowel Bacterial Overgrowth, Sudden Infant Death Syndrome (SIDS), Systemic Lupus Erythematosus, Ulcerative Colitis, Upper Abdominal FBD, Vasculitic Disorders, Viral Gastroenteritis, pre-diabetic syndrome, type I diabetes, type II diabetes, depression, schizophrenia, and a mood disorder.

In another aspect, this disclosure provides a method for treating a disorder or condition in a subject in need thereof, comprising administering to the subject a therapeutically effective amount of a pharmaceutical composition or a previously frozen fecal microbe composition disclosed herein, wherein the disorder or condition is selected from the group consisting of recurrent C. diff infection, autism spectrum disorder (ASD), constipation predominant functional bowel disease (FBD), pain predominant FBD, upper abdominal FBD, non-ulcer dyspepsia (NUD), gastro-oesophageal reflux, indeterminate colitis, microscopic colitis, pseudomembranous colitis, viral gastroenteritis, Norwalk viral gastroenteritis, rotavirus gastroenteritis, AIDS related gastroenteritis, non rheumatoid factor positive arthritis, Lyme disease, systemic lupus, idiopathic thrombocytopenic purpura, Sjogren's syndrome, haemolytic uremic syndrome or scleroderma, Gillain-Barre syndrome, Chronic Inflammatory Demyelinating Polyneuropathy, chronic depression, schizophrenia, psychotic disorders, manic depressive illness, Asbergers syndrome, Rett syndrome, attention deficit hyperactivity disorder (ADHD), attention deficit disorder (ADD), sudden infant death syndrome (SIDS), and anorexia nervosa.

In another aspect, this disclosure provides a method for treating a disorder or condition in a subject in need thereof, comprising administering to the subject a therapeutically effective amount of a pharmaceutical composition or a previously frozen fecal microbe composition disclosed herein, wherein the disorder or condition is selected from the group consisting of recurrent or primary C. diff infection, autism spectrum disorder (ASD), ulcerative colitis, Crohn's disease, and irritable bowel syndrome.

In one aspect, a fecal microbiota preparation comprises a donor's entire or substantially complete microbiota. In one aspect, a fecal microbiota preparation comprises a non-selected fecal microbiota. In another aspect, a fecal microbiota preparation comprises an isolated or purified population of live non-pathogenic fecal bacteria. In a further aspect, a fecal microbiota preparation comprises a non-selected and substantially complete fecal microbiota preparation from a single donor.

In one aspect, bacterial cell viability is measured by using imaging assays that measure membrane permeability. A combination of membrane permeant and impermeant DNA dyes stains are used (e.g., intact cells stained green and dead cells stained red). In one aspect, a SYTO BC dye and propidium idodide are used to stain and differentiate live and dead bacteria. See Example 2. In one aspect, SYTO 9 and propidium idodide are used to stain and differentiate live and dead bacteria. See Stocks, Cytometry A. 2004 October; 61(2):189-95. In another aspect, live cell determination is combined with fluorescent Gram staining. In another aspect, the number of viable bacterial cells in a sample is assessed by a colorimetric method, e.g., a Dojindo's Microbial Viability Assay Kit-WST.

In one aspect, bacterial cell viability assessed by counting the number of colonies on an agar plate is the standard method for determining the number of viable bacterial cells in samples. In another aspect, cell viability is evaluated via molecular viability analyses, e.g., a PCR-based approach, which can differentiate nucleic acids associated with viable cells from those associated with non-viable cells. See Cangelosi and Mescheke, Appl Environ Microbiol. 2014 October; 80(19): 5884-5891.

In one aspect, a therapeutic composition comprises a cryoprotectant. In another aspect, a cryoprotectant comprises, consists essentially of, or consists of polyethylene glycol, skim milk, erythritol, arabitol, sorbitol, glucose, fructose, alanine, glycine, proline, sucrose, lactose, ribose, trehalose, dimethyl sulfoxide (DMSO), glycerol, or a combination thereof. In an aspect of the present disclosure, a cryoprotectant can be selected from the group comprising 5% Sucrose; 10% Sucrose; 10% Skim milk; 10% Trehalose with 2.5% sucrose; 5% Trehalose with 2.5% sucrose; 5% Mannitol; 5% Mannitol with 0.1% Polysorbate 80; 10% Mannitol; 10% Mannitol with 0.1% Polysorbate 80; 5% Trehalose; 5% Trehalose with 0.1% Polysorbate 80; 10% Trehalose; and 10% Trehaolse with 0.1% Polysorbate 80.

In another aspect, a therapeutic composition comprises a lyoprotectant. In one aspect, the same substance or the same substance combination is used as both a cryoprotectant and a lyoprotectant. Exemplary lyoprotectants include sugars such as sucrose or trehalose; an amino acid such as monosodium glutamate or histidine; a methylamine such as betaine; a lyotropic salt such as magnesium sulfate; a polyol such as trihydric or higher sugar alcohols, e.g. glycerin, erythritol, glycerol, arabitol, xylitol, sorbitol, and mannitol; propylene glycol; polyethylene glycol; Pluronics; and combinations thereof. In one aspect, a lyoprotectant is a non-reducing sugar, such as trehalose or sucrose. In one aspect, a cryoprotectant or a lyoprotectant consists essentially of, or consists of, one or more substances mentioned in this paragraph and the paragraph above.

In one aspect, a cryoprotectant or a lyoprotectant comprise an intracellular agent, e.g., DMSO, Glycerol, or PEG, which penetrates inside the cell preventing the formation of ice crystals that could result in membrane rupture. In another aspect, a cryoprotectant or a lyoprotectant comprise an extracellular agent, e.g., sucrose, trehalose, or dextrose, which does not penetrate into the cell membrane but acts to improve the osmotic imbalance that occurs during freezing.

In one aspect, a lyophilized formulation comprises trehalose. In one aspect, a lyophilized formulation comprises 2% to 30%, 3% to 25%, 4% to 20%, 5% to 15%, 6% to 10%, 2% to 30%, 2% to 25%, 2% to 20%, 2% to 15%, or 2% to 10% trehalose. In one aspect, a lyophilized formulation comprises at least 2%, 3%, 4%, 5%, 6%, 7%, 8%, 9%, 10%, or 15% trehalose. In one aspect, a lyophilized formulation comprises at most 2%, 3%, 4%, 5%, 6%, 7%, 8%, 9%, 10%, or 15% trehalose. In one aspect, a lyophilized formulation comprises about 5% trehalose. In one aspect, a lyophilized formulation comprises trehalose and sucrose. In one aspect, a lyophilized formulation comprises between about 8% to 12% trehalose with between about 1.5% to 3.5% sucrose and between about 0.5% to 1.5% NaCl.

In one aspect, the preparation of a fecal microbiota preparation involves a treatment selected from the group consisting of ethanol treatment, detergent treatment, heat treatment, irradiation, and sonication, or a combination thereof. In one aspect, the preparation of a fecal microbiota preparation involves no treatment selected from the group consisting of ethanol treatment, detergent treatment, heat treatment, irradiation, and sonication. In one aspect, the preparation of a fecal microbiota preparation involves a separation step selected from the group consisting of filtering, sieving, density gradients, filtration, chromatography, and a combination thereof. In one aspect, the preparation of a fecal microbiota preparation does not require one or more separation steps selected from the group consisting of filtering, sieving, density gradients, filtration, and chromatography. In one aspect, a fecal microbiota preparation is substantially free of non-living matter. In one aspect, a fecal microbiota preparation is substantially free of acellular material selected from the group consisting of residual fiber, DNA, viral coat material, and non-viable material. In one aspect, a fecal microbiota preparation is substantially free of eukaryotic cells from the fecal microbiota's donor.

In one aspect, a pharmaceutical composition provided here, after at least 12 weeks of storage at ambient temperature or lower, is effective for treating one or more disorders selected from the group consisting of recurrent or primary C. diff infection, autism spectrum disorder (ASD), ulcerative colitis, Crohn's disease, and irritable bowel syndrome. In another aspect, a pharmaceutical composition remains effective after at least 4, 8, 10, 16, 20, 24, 30, 40, 50, 60, 70, 80 or 100 weeks of storage at ambient temperature or lower.

In one aspect, the present disclosure provides a method for treating a disorder (e.g., C. difficile infection, autism spectrum disorder (ASD), ulcerative colitis, Crohn's disease, or another indication listed herein) in a subject in need thereof, where the method comprises administering to the subject a pharmaceutically active dose of a therapeutic composition described herein. In one aspect, the present disclosure provides a method for treating a disorder (e.g., C. difficile infection, ASD, ulcerative colitis, or Crohn's disease) in a subject in need thereof, where the method comprises administering daily to the subject a pharmaceutically active dose of a therapeutic composition described herein. In one aspect, a therapeutic composition is administered to a patient in need thereof at least once daily for at least two consecutive days. In one aspect, a therapeutic composition is administered at least once daily for at least 3, 4, 5, 6, 7, 8, 9, 10, 11, 12, 13, 14, or 15 consecutive days. In another aspect, a therapeutic composition is administered at least once daily for at least 1, 2, 3, 4, 5, 6, 7, 8, 9, 10, 11, or 12 consecutive weeks. In another aspect, a therapeutic composition is administered at least twice, three times, four times, or five times per week for at least 1, 2, 3, 4, 5, 6, 7, 8, 9, 10, 11, or 12 consecutive weeks. In one aspect, a therapeutic composition is administered at least once daily for at most 4, 5, 6, 7, 8, 9, 10, 11, 12, 13, 14, 15, 16, 17, 18, 19, or 20 consecutive days or weeks. In another aspect, a therapeutic composition is administered at least once daily for at most 1, 2, 3, 4, 5, 6, 7, 8, 9, 10, 11, or 12 consecutive weeks or months. In a further aspect, a therapeutic composition is administered at least once for at least 1, 2, 3, 4, 5, 6, 7, 8, 9, 10, 11, or 12 consecutive months or years, chronically for a subject's entire life span, or an indefinite period of time.

In one aspect, a therapeutic composition is administered to a patient in need thereof at least twice daily for at least two consecutive days. In one aspect, a therapeutic composition is administered at least twice daily for at least 3, 4, 5, 6, 7, 8, 9, 10, 11, 12, 13, 14, or 15 consecutive days. In another aspect, a therapeutic composition is administered at least twice daily for at least 1, 2, 3, 4, 5, 6, 7, 8, 9, 10, 11, or 12 consecutive weeks. In one aspect, a therapeutic composition is administered at least twice daily for at most 4, 5, 6, 7, 8, 9, 10, 11, 12, 13, 14, 15, 16, 17, 18, 19, or 20 consecutive days or week. In another aspect, a therapeutic composition is administered at least twice daily for at most 1, 2, 3, 4, 5, 6, 7, 8, 9, 10, 11, or 12 consecutive weeks or months. In a further aspect, a therapeutic composition is administered at least twice for at least 1, 2, 3, 4, 5, 6, 7, 8, 9, 10, 11, or 12 consecutive months or years, chronically for a subject's entire life span, or an indefinite period of time.

In one aspect, a therapeutic composition is administered to a patient in need thereof at least three times daily for at least two consecutive days. In one aspect, a therapeutic composition is administered at least three times daily for at least 3, 4, 5, 6, 7, 8, 9, 10, 11, 12, 13, 14, or 15 consecutive days. In another aspect, a therapeutic composition is administered at least three times daily for at least 1, 2, 3, 4, 5, 6, 7, 8, 9, 10, 11, or 12 consecutive weeks. In one aspect, a therapeutic composition is administered at least three times daily for at most 4, 5, 6, 7, 8, 9, 10, 11, 12, 13, 14, 15, 16, 17, 18, 19, or 20 consecutive days or weeks. In another aspect, a therapeutic composition is administered at least three times daily for at most 1, 2, 3, 4, 5, 6, 7, 8, 9, 10, 11, or 12 consecutive weeks or months. In a further aspect, a therapeutic composition is administered at least three times for at least 1, 2, 3, 4, 5, 6, 7, 8, 9, 10, 11, or 12 consecutive months or years, chronically for a subject's entire life span, or an indefinite period of time.

In one aspect, the present disclosure provides a method for treating a disorder (e.g., C. difficile infection, ASD, ulcerative colitis, or Crohn's disease) in a subject in need thereof, where the method comprises administering orally to the subject a pharmaceutically active dose of a therapeutic composition comprising live, non-pathogenic, synthetic bacterial mixture or live, non-pathogenic, purified or extracted, fecal microbiota in a lyophilized formulation described herein, where the dose is administered at a dosing schedule of at least once or twice daily for at least three consecutive days or weeks. In another aspect, a dose is administered at least once, twice, or three times daily for a period between 1 and 12 weeks, between 2 and 12 weeks, between 3 and 12 weeks, between 4 and 12 weeks, between 5 and 12 weeks, between 6 and 12 weeks, between 7 and 12 weeks, between 8 and 12 weeks, between 9 and 12 weeks, between 10 and 12 weeks, between 1 and 2 weeks, between 2 and 3 weeks, between 3 and 4 weeks, between 4 and 5 weeks, between 5 and 6 weeks, between 6 and 7 weeks, between 7 and 8 weeks, between 8 and 9 weeks, between 9 and 10 weeks, or between 10 and 11 weeks.

In one aspect, the present disclosure provides a method for treating a disorder (e.g., C. difficile infection, ASD, ulcerative colitis, or Crohn's disease) in a subject in need thereof by administering a pharmaceutical composition described herein, where the method comprises a first dosing schedule followed by a second dosing schedule. In one aspect, a first dosing schedule comprises a treatment or induction dose. In one aspect, a first dosing schedule comprises a continuous dosing schedule. In another aspect, a second dosing schedule comprises a maintenance dose lower than or equal to a pharmaceutically active dose of a first dosing schedule. In another aspect, a second dosing schedule lasts for at least about 2, 4, 6, 8, 10, 12, 18, 24, 36, 48, 72, or 96 months. In one aspect, a second dosing schedule lasts permanently, for a treated subject's entire life span, or an indefinite period of time. In one aspect, a second dosing schedule is a continuous dosing schedule. In another aspect, a second dosing schedule is an intermittent dosing schedule. In a further aspect, a second dosing schedule is an intermittent dosing schedule comprising a treatment period of at least 1, 2, 3, 4, 5, 6, 7, 8, 9, 10, 11, 12, 13, or 14 days followed by a resting period of at least 1, 2, 3, 4, 5, 6, 7, 8, 9, 10, 11, 12, 13, or 14 days. In another aspect, a second dosing schedule comprises administering a second dose (e.g., a maintenance dose) every other day, every two days, or every 3, 4, 5, 6, 7, 8 days. In another aspect, a maintenance dose is administered for an extended period of time with or without titration (or otherwise changing the dosage or dosing schedule). In one aspect, the interval between a first and a second dosing schedule is at least about 1, 2, 3, 4, 5, 6, 7, 8, 9, 10, 11, or 12 weeks. In another aspect, a second dosing schedule (e.g., a maintenance dose) comprises a dosage about 2, 5, 10, 50, 100, 200, 400, 800, 1000, 5000 or more fold lower than the dosage used in a first dosing schedule (e.g., an initial treatment dose). In another aspect, a second dosing schedule (e.g., a maintenance dosing schedule) has an equal or lower dosing frequency than a first dosing schedule (e.g., an initial treatment dosing schedule). In another aspect, a second dosing schedule (e.g., a maintenance dosing schedule) has a higher dosing interval than a first dosing schedule (e.g., an initial treatment dosing schedule).

In one aspect, a first or second dosing schedule used in a method can be once-a-week, twice-a-week, or thrice-a-week. The term “once-a-week” means that a dose is administered once in a week, preferably on the same day of each week. “Twice-a-week” means that a dose is administered two times in a week, preferably on the same two days of each weekly period. “Thrice-a-week” means that a dose is administered three times in a week, preferably on the same three days of each weekly period.

In one aspect, a subject being treated is a subject already with a disorder (e.g., ulcerative colitis or Crohn's disease). Administration of a disclosed therapeutic composition to a clinically asymptomatic human subject who is genetically predisposed or prone to a disorder (e.g., ulcerative colitis or Crohn's disease) is also useful in preventing or inhibiting the onset of clinical symptoms. A human subject genetically predisposed or prone to ulcerative colitis can be a human subject having a close family member or relative exhibiting or having suffered a disorder (e.g., ulcerative colitis or Crohn's disease). In another aspect, a subject being treated is a subject in which ulcerative colitis is to be prevented or inhibited. In another aspect, a subject being treated is predisposed or susceptible to a disorder (e.g., ulcerative colitis or Crohn's disease). In another aspect, a subject being treated is a subject diagnosed as having a disorder (e.g., ulcerative colitis or Crohn's disease). In one aspect, a subject being treated is a patient in need thereof.

In one aspect, a subject being treated is a human patient. In one aspect, a patient is a male patient. In one aspect, a patient is a female patient. In one aspect, a patient is a premature newborn. In one aspect, a patient is a term newborn. In one aspect, a patient is a neonate. In one aspect, a patient is an infant. In one aspect, a patient is a toddler. In one aspect, a patient is a young child. In one aspect, a patient is a child. In one aspect, a patient is an adolescent. In one aspect, a patient is a pediatric patient. In one aspect, a patient is a geriatric patient. In one aspect, a human patient is a child patient below about 18, 15, 12, 10, 8, 6, 4, 3, 2, or 1 year old. In another aspect, a human patient is an adult patient. In another aspect, a human patient is an elderly patient. In a further aspect, a human patient is a patient above about 30, 35, 40, 45, 50, 55, 60, 65, 70, 75, 80, 85, 90, or 95 years old. In another aspect, a patient is about between 1 and 5, between 2 and 10, between 3 and 18, between 21 and 50, between 21 and 40, between 21 and 30, between 50 and 90, between 60 and 90, between 70 and 90, between 60 and 80, or between 65 and 75 years old. In one aspect, a patient is a young old patient (65-74 years). In one aspect, a patient is a middle old patient (75-84 years). In one aspect, a patient is an old patient (>85 years).

In one aspect, a method comprises administering a therapeutic composition orally, by enema, or via rectal suppository. In one aspect, a pharmaceutical composition is formulated as a geltab, pill, microcapsule, capsule, or tablet. In one aspect, a therapeutic composition is formulated as an enteric coated capsule or microcapsule, acid-resistant capsule or microcapsule, or formulated as part of or administered together with a food, a food additive, a dairy-based product, a soy-based product or a derivative thereof, a jelly, or a yogurt. In another aspect, a therapeutic composition is formulated as an acid-resistant enteric coated capsule. A therapeutic composition can be provided as a powder for sale in combination with a food or drink. A food or drink can be a dairy-based product or a soy-based product. In another aspect, a food or food supplement contains enteric-coated and/or acid-resistant microcapsules containing a therapeutic composition.

In an aspect, a therapeutic composition comprises a liquid culture. In another aspect, a therapeutic composition is lyophilized, pulverized and powdered. It may then be infused, dissolved such as in saline, as an enema. Alternatively the powder may be encapsulated as enteric-coated and/or acid-resistant capsules for oral administration. These capsules may take the form of enteric-coated and/or acid-resistant microcapsules. A powder can preferably be provided in a palatable form for reconstitution for drinking or for reconstitution as a food additive. In a further aspect, a food is yogurt. In one aspect, a powder may be reconstituted to be infused via naso-duodenal infusion. In another aspect, therapeutic compositions are designed for targeted delivery of an active ingredient into the distal small bowel or the colon.

In another aspect, a therapeutic composition is in a liquid, frozen, freeze-dried, spray-dried, foam-dried, lyophilized, or powder formulation. In a further aspect, a therapeutic composition is formulated as a delayed or gradual enteric release form. In another aspect, a therapeutic composition comprises an excipient, a saline, a buffer, a buffering agent, or a fluid-glucose-cellobiose agar (RGCA) media.

In one aspect, a therapeutic composition further comprises an acid suppressant, an antacid, an H2 antagonist, a proton pump inhibitor or a combination thereof. In one aspect, a therapeutic composition is substantially free of non-living matter. In another aspect, a therapeutic composition is substantially free of acellular material selected from the group consisting of residual fiber, DNA, viral coat material, and non-viable material.

In one aspect, a therapeutic composition also comprises or is supplemented with a prebiotic nutrient selected from the group consisting of polyols, fructooligosaccharides (FOSs), oligofructoses, inulins, galactooligosaccharides (GOSs), xylooligosaccharides (XOSs), polydextroses, monosaccharides, tagatose, and/or mannooligosaccharides.

In one aspect, a method further comprises pretreating a subject with an antibiotic composition prior to administering a therapeutic bacterial or microbiota composition. In one aspect, an antibiotic composition comprises an antibiotic selected from the group consisting of rifabutin, clarithromycin, clofazimine, vancomycin, rifampicin, nitroimidazole, chloramphenicol, and a combination thereof. In another aspect, an antibiotic composition comprises an antibiotic selected from the group consisting of rifaximin, rifamycin derivative, rifampicin, rifabutin, rifapentine, rifalazil, bicozamycin, aminoglycoside, gentamycin, neomycin, streptomycin, paromomycin, verdamicin, mutamicin, sisomicin, netilmicin, retymicin, kanamycin, aztreonam, aztreonam macrolide, clarithromycin, dirithromycin, roxithromycin, telithromycin, azithromycin, bismuth subsalicylate, vancomycin, streptomycin, fidaxomicin, amikacin, arbekacin, neomycin, netilmicin, paromomycin, rhodostreptomycin, tobramycin, apramycin, and a combination thereof. In a further aspect, a method further comprises pretreating a subject with an anti-inflammatory drug prior to administration of a therapeutic bacterial or microbiota composition.

In one aspect, every about 200 mg of a pharmaceutical composition comprises a pharmacologically active dose. In one aspect, every about 75, 100, 125, 150, 175, 200, 250, 300, 350, 400, 450, 500, 750, 1000, 1500, or 2000 mg of a pharmaceutical composition comprises a pharmacologically active dose.

In one aspect, a pharmaceutically active or therapeutic effective dose comprises at least about 10⁵, 10⁶, 10⁷, 10⁸, 10⁹, 10¹⁰, 10¹¹, 10¹², or 10¹³ cfu. In another aspect, a pharmaceutically active therapeutic effective dose comprises at most about 10⁵, 10⁶, 10⁷, 10⁸, 10⁹, 10¹⁰, 10¹¹, 10¹², 10¹³, 10¹⁴, or 10¹⁵ cfu. In a further aspect, a pharmacologically active therapeutic effective dose is selected from the group consisting of from 10⁸ cfu to 10¹⁴ cfu, from 10⁹ cfu to 10¹³ cfu, from 10¹⁰ cfu to 10¹² cfu, from 10⁹ cfu to 10¹⁴ cfu, from 10⁹ cfu to 10¹² cfu, from 10⁹ cfu to 10¹¹ cfu, from 10⁹ cfu to 10¹⁰ cfu, from 10¹⁰ cfu to 10¹⁴ cfu, from 10¹⁰ cfu to 10¹³ cfu, from 10¹¹ cfu to 10¹⁴ cfu, from 10¹¹ cfu to 10¹³ cfu, from 10¹² cfu to 10¹⁴ cfu, and from 10¹³ cfu to 10¹⁴ cfu.

In one aspect, a pharmaceutically active or therapeutic effective dose comprises at least about 10⁵, 10⁶, 10⁷, 10⁸, 10⁹, 10¹⁰, 10¹¹, 10¹², or 10¹³ cells or spores. In another aspect, a pharmaceutically active or therapeutic effective dose comprises at most about 10⁵, 10⁶, 10⁷, 10⁸, 10⁹, 10¹⁰, 10¹¹, 10¹², or 10¹³ total cells or spores. In a further aspect, a pharmacologically active or therapeutic effective dose is selected from the group consisting of from 10⁸ to 10¹⁴, from 10⁹ to 10¹³, from 10¹⁰ to 10¹², from 10⁹ to 10¹⁴, from 10⁹ to 10¹², from 10⁹ to 10¹¹, from 10⁹ to 10¹⁰, from 10¹⁰ to 10¹⁴, from 10¹⁰ to 10¹³, from 10¹¹ to 10¹⁴, from 10¹¹ to 10¹³, from 10¹² to 10¹⁴, and from 10¹³ to 10¹⁴ cells or spores. In an aspect, the pharmaceutically active or therapeutic effective dose cell count is directed to live cells.

In one aspect, a therapeutic composition comprises one or more, two or more, three or more, four or more, or five or more isolated, purified, or cultured microorganisms selected from the group consisting of Clostridium, Bacillus, Collinsella, Bacteroides, Eubacterium, Fusobacterium, Propionibacterium, Lactobacillus, Ruminococcus, Escherichia coli, Gemmiger, Desulfomonas, Peptostreptococcus, Bifidobacterium, Coprococcus, Dorea, and Monilia.

In one aspect, a fecal microbiota preparation described herein comprises a purified or reconstituted fecal bacterial mixture. In one aspect, a fecal microbiota preparation comprises one or more, one or more, two or more, three or more, four or more, or five or more live fecal microorganisms are selected from the group consisting of Acidaminococcus, Akkermansia, Alistipes, Anaerotruncus, Bacteroides, Bifidobacterium, Blautia, Butyrivibrio, Clostridium, Collinsella, Coprococcus, Corynebacterium, Dorea, Enterococcus, Escherichia, Eubacterium, Faecalibacterium, Haemophilus, Holdemania, Lactobacillus, Moraxella, Parabacteroides, Prevotella, Propionibacterium, Raoultella, Roseburia, Ruminococcus, Staphylococcus, Streptococcus, Subdoligranulum, and Veillonella. In one aspect, a fecal microbiota preparation comprises one or more, one or more, two or more, three or more, four or more, or five or more live fecal microorganisms selected from the group consisting of Bacteroides fragilis ssp. vulgatus, Collinsella aerofaciens, Bacteroides fragilis ssp. thetaiotaomicron, Peptostreptococcus productus II, Parabacteroides distasonis, Faecalibacterium prausnitzii, Coprococcus eutactus, Peptostreptococcus productus I, Ruminococcus bromii, Bifdobacterium adolescentis, Gemmiger formicilis, Bifdobacterium longum, Eubacterium siraeum, Ruminococcus torques, Eubacterium rectale, Eubacterium eligens, Bacteroides eggerthii, Clostridium leptum, Bacteroides fragilis ssp. A, Eubacterium biforme, Bifdobacterium infantis, Eubacterium rectale, Coprococcus comes, Pseudoflavonifractor capillosus, Ruminococcus albus, Dorea formicigenerans, Eubacterium halii, Eubacterium ventriosum I, Fusobacterium russi, Ruminococcus obeum, Eubacterium rectale, Clostridium ramosum, Lactobacillus leichmannii, Ruminococcus callidus, Butyrivibrio crossotus, Acidaminococcus fermentans, Eubacterium ventriosum, Bacteroides fragilis ssp. fragilis, Coprococcus catus, Aerostipes hadrus, Eubacterium cylindroides, Eubacterium ruminantium, Staphylococcus epidermidis, Eubacterium limosum, Tissirella praeacuta, Fusobacterium mortiferum I, Fusobacterium naviforme, Clostridium innocuum, Clostridium ramosum, Propionibacterium acnes, Ruminococcus flavefaciens, Bacteroides fragilis ssp. ovatus, Fusobacterium nucleatum, Fusobacterium mortiferum, Escherichia coli, Gemella morbillorum, Finegoldia magnus, Streptococcus intermedius, Ruminococcus lactaris, Eubacterium tenue, Eubacterium ramulus, Bacteroides clostridiiformis ssp. clostridliformis, Bacteroides coagulans, Prevotella oralis, Prevotella ruminicola, Odoribacter splanchnicus, and Desuifomonas pigra.

In one aspect, a fecal microbiota preparation lacks or is substantially devoid of one or more, two or more, three or more, four or more, or five or more live fecal microorganisms selected from the group consisting of Acidaminococcus, Akkermansia, Alistipes, Anaerotruncus, Bacteroides, Biidobacterium, Blautia, Butyrivibrio, Clostridium, Collinsella, Coprococcus, Corynebacterium, Dorea, Enterococcus, Escherichia, Eubacterium, Faecalibacterium, Haemophilus, Holdemania, Lactobacillus, Moraxella, Parabacteroides, Prevotella, Propionibacterium, Raoultella, Roseburia, Ruminococcus, Staphylococcus, Streptococcus, Subdoligranulum, and Veillonella. In one aspect, a fecal microbiota preparation lacks or is substantially devoid of one or more, two or more, three or more, four or more, or five or live more fecal microorganisms are selected from the group consisting of Bacteroides fragilis ssp. vulgatus, Collinsella aerofaciens, Bacteroides fragilis ssp. thetaiotaomicron, Peptostreptococcus productus II, Parabacteroides distasonis, Faecalibacterium prausnitzii, Coprococcus eutactus, Peptostreptococcus productus I, Ruminococcus bromii, Bifidobacterium adolescentis, Gemmiger formicilis, Bifidobacterium longum, Eubacterium siraeum, Ruminococcus torques, Eubacterium rectale, Eubacterium eligens, Bacteroides eggerthii, Clostridium leptum, Bacteroides fragilis ssp. A, Eubacterium biforme, Bifidobacterium infantis, Eubacterium rectale, Coprococcus comes, Pseudoflavonifractor capillosus, Ruminococcus albus, Dorea formicigenerans, Eubacterium halii, Eubacterium ventriosum I, Fusobacterium russi, Ruminococcus obeum, Eubacterium rectale, Clostridium ramosum, Lactobacillus leichmannii, Ruminococcus callidus, Butyrivibrio crossotus, Acidaminococcus fermentans, Eubacterium ventriosum, Bacteroides fragilis ssp. fragilis, Coprococcus catus, Aerostipes hadrus, Eubacterium cylindroides, Eubacterium ruminantium, Staphylococcus epidermidis, Eubacterium limosum, Tissirella praeacuta, Fusobacterium mortiferum I, Fusobacterium naviforme, Clostridium innocuum, Clostridium ramosum, Propionibacterium acnes, Ruminococcus flavefaciens, Bacteroides fragilis ssp. ovatus, Fusobacterium nucleatum, Fusobacterium mortiferum, Escherichia coli, Gemella morbillorum, Finegoldia magnus, Streptococcus intermedius, Ruminococcus lactaris, Eubacterium tenue, Eubacterium ramulus, Bacteroides clostridiiformis ssp. clostridliformis, Bacteroides coagulans, Prevotella oralis, Prevotella ruminicola, Odoribacter splanchnicus, and Desuifomonas pigra.

In another aspect, a therapeutic composition comprises a fecal microbiota further supplemented, spiked, or enhanced with a fecal microorganism. In one aspect, a fecal microbiota is supplemented with a non-pathogenic (or with attenuated pathogenicity) bacterium of Clostridium, Collinsella, Dorea, Ruminococcus, Coprococcus, Prevotella, Veillonella, Bacteroides, Baccillus, or a combination thereof. In another aspect, a therapeutic composition comprises a fecal microbiota further supplemented, spiked, or enhanced with a species of Veillonellaceae, Firmicutes, Gammaproteobacteria, Bacteroidetes, or a combination thereof. In another aspect, a therapeutic composition comprises a fecal microbiota further supplemented with fecal bacterial spores. In one aspect, fecal bacterial spores are Clostridium spores, Bacillus spores, or both.

In an aspect, a therapeutic composition comprises a fecal microbiota from a subject selected from the group consisting of a human, a bovine, a dairy calf, a ruminant, an ovine, a caprine, or a cervine. In another aspect, a therapeutic composition can be administered to a subject selected from the group consisting of a human, a bovine, a dairy calf, a ruminant, an ovine, a caprine, or a cervine. In an aspect, a therapeutic composition is substantially or nearly odourless.

In an aspect, a therapeutic composition provided here comprises a fecal microbiota comprising a Shannon Diversity Index of greater than or equal to 0.3, greater than or equal to 0.4, greater than or equal to 0.5, greater than or equal to 0.6, greater than or equal to 0.7, greater than or equal to 0.8, greater than or equal to 0.9, greater than or equal to 1.0, greater than or equal to 1.1, greater than or equal to 1.2, greater than or equal to 1.3, greater than or equal to 1.4, greater than or equal to 1.5, greater than or equal to 1.6, greater than or equal to 1.7, greater than or equal to 1.8, greater than or equal to 1.9, greater than or equal to 2.0, greater than or equal to 2.1, greater than or equal to 2.2, greater than or equal to 2.3, greater than or equal to 2.4, greater than or equal to 2.5, greater than or equal to 3.0, greater than or equal to 3.1, greater than or equal to 3.2, greater than or equal to 3.3, greater than or equal to 3.4, greater than or equal to 3.5, greater than or equal to 3.6, greater than or equal to 3.7, greater than or equal to 3.8, greater than or equal to 3.9, greater than or equal to 4.0, greater than or equal to 4.1, greater than or equal to 4.2, greater than or equal to 4.3, greater than or equal to 4.4, greater than or equal to 4.5, or greater than or equal to 5.0. In another aspect, a therapeutic composition comprises fecal microbiota comprising a Shannon Diversity Index of between 0.1 and 3.0, between 0.1 and 2.5, between 0.1 and 2.4, between 0.1 and 2.3, between 0.1 and 2.2, between 0.1 and 2.1, between 0.1 and 2.0, between 0.4 and 2.5, between 0.4 and 3.0, between 0.5 and 5.0, between 0.7 and 5.0, between 0.9 and 5.0, between 1.1 and 5.0, between 1.3 and 5.0, between 1.5 and 5.0, between 1.7 and 5.0, between 1.9 and 5.0, between 2.1 and 5.0, between 2.3 and 5.0, between 2.5 and 5.0, between 2.7 and 5.0, between 2.9 and 5.0, between 3.1 and 5.0, between 3.3 and 5.0, between 3.5 and 5.0, between 3.7 and 5.0, between 31.9 and 5.0, or between 4.1 and 5.0. In one aspect, a Shannon Diversity Index is calculated at the phylum level. In another aspect, a Shannon Diversity Index is calculated at the family level. In one aspect, a Shannon Diversity Index is calculated at the genus level. In another aspect, a Shannon Diversity Index is calculated at the species level. In a further aspect, a therapeutic composition comprises a preparation of flora in proportional content that resembles a normal healthy human fecal flora.

In a further aspect, a therapeutic composition comprises fecal bacteria from at least 1, 2, 3, 4, 5, 6, 7, 8, 9, or 10 different families. In another aspect, a therapeutic composition comprises fecal bacteria from at least 11, 12, 13, 14, 15, 16, 17, 18, 19, or 20 different families. In yet another aspect, a therapeutic composition comprises fecal bacteria from at least 21, 22, 23, 24, 25, 26, 27, 28, 29, or 30 different families. In a further aspect, a therapeutic composition comprises fecal bacteria from at least 31, 32, 33, 34, 35, 36, 37, 38, 39, or 40 different families. In another aspect, a therapeutic composition comprises fecal bacteria from at least 41, 42, 43, 44, 45, 46, 47, 48, 49, or 50 different families. In another aspect, a therapeutic composition comprises fecal bacteria from between 1 and 10, between 10 and 20, between 20 and 30, between 30 and 40, between 40 and 50 different families. In an aspect, a therapeutic composition provided here comprises a fecal microbiota comprising no greater than 0.05%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, or 10% weight non-living material/weight biological material. In another aspect, a therapeutic composition provided here comprises a fecal microbiota comprising no greater than 20%, 25%, 30%, 35%, 40%, 45%, 50%, 55%, 60%, 65%, 70%, 75%, 80%, 85%, 90%, or 95% weight non-living material/weight biological material. In another aspect, a therapeutic composition provided here comprises, consists of, or consists essentially of, particles of non-living material and/or particles of biological material of a fecal sample that passes through a sieve, a column, or a similar filtering device having a sieve, exclusion, or particle filter size of 2.0 mm, 1.0 mm, 0.5 mm, 0.25 mm, 0.212 mm, 0.180 mm, 0.150 mm, 0.125 mm, 0.106 mm, 0.090 mm, 0.075 mm, 0.063 mm, 0.053 mm, 0.045 mm, 0.038 mm, 0.032 mm, 0.025 mm, 0.020 mm, 0.01 mm, 0.004 mm, 0.002 mm, 0.001 mm or 0.0005 mm. “Non-living material” does not include an excipient, e.g., a pharmaceutically inactive substance, such as a cryoprotectant, added to a processed fecal material. “Biological material” refers to the living material in fecal material, and includes microbes including prokaryotic cells, such as bacteria and archaea (e.g., living prokaryotic cells and spores that can sporulate to become living prokaryotic cells), eukaryotic cells such as protozoa and fungi, and viruses. In one aspect, “biological material” refers to the living material, e.g., the microbes, eukaryotic cells, and viruses, which are present in the colon of a normal healthy human. In an aspect, a therapeutic composition provided or comprises an extract of human feces where the composition is substantially odorless. In an aspect, a therapeutic composition provided or comprises fecal material or a fecal floral preparation in a lyophilized, crude, semi-purified or purified formulation.

In an aspect, a fecal microbiota in a therapeutic composition comprises highly refined or purified fecal flora, e.g., substantially free of non-floral fecal material. In an aspect, a fecal microbiota can be further processed, e.g., to undergo microfiltration before, after, or before and after sieving. In another aspect, a highly purified fecal microbiota product is ultra-filtrated to remove large molecules but retain the therapeutic microflora, e.g., bacteria.

In another aspect, a fecal microbiota in a therapeutic composition used herein comprises or consists essentially of a substantially isolated or a purified fecal flora or entire (or substantially entire) microbiota that is (or comprises) an isolate of fecal flora that is at least about 40%, 45%, 50%, 55%, 60%, 65%, 70%, 75%, 80%, 85%, 90%, 91%, 92%, 93%, 94%, 95%, 96%, 97%, 98%, 99%, 99.5%, 99.6%, 99.7%, 99.8% or 99.9% isolated or pure, or having no more than about 0.10%, 0.2%, 0.3%, 0.4%, 0.5%, 0.6%, 0.7%, 0.8%, 0.9%, 1.0%, 2%, 5%, 10%, 15%, 20%, 25%, 30%, 35%, 40%, 45%, 50%, 55% or more non-fecal floral material; or, a substantially isolated, purified, or substantially entire microbiota as described in Sadowsky et al, WO 2012/122478 A1, or as described in Borody et al., WO 2012/016287 A2. In one aspect, a fecal microbiota preparation comprises a weight ratio between fecal-derived non-living material and fecal-derived biological material of no greater than about 0.1%, 0.2%, 0.3%, 0.4%, 0.5%, 0.6%, 0.7%, 0.8%, 0.9%, 1%, 2%, 5%, 8%, 10%, 15%, 20%, 30%, 40%, or 50%.

In an aspect, a fecal microbiota in a therapeutic composition comprises a donor's substantially entire or non-selected fecal microbiota, reconstituted fecal material, or synthetic fecal material. In another aspect, the fecal microbiota in a therapeutic composition comprises no antibiotic resistant population. In another aspect, a therapeutic composition comprises a fecal microbiota and is largely free of extraneous matter (e.g., non-living matter including acellular matter such as residual fiber, DNA, RNA, viral coat material, non-viable material; and living matter such as eukaryotic cells from the fecal matter's donor).

In an aspect, a fecal microbiota in a therapeutic composition used herein is derived from disease-screened fresh homologous feces or equivalent freeze-dried and reconstituted feces. In an aspect, a fresh homologous feces does not include an antibiotic resistant population. In another aspect, a fecal microbiota in a therapeutic composition is derived from a synthetic fecal composition. In an aspect, a synthetic fecal composition comprises a preparation of viable flora which preferably in proportional content, resembles normal healthy human fecal flora which does not include antibiotic resistant populations. Suitable microorganisms may be selected from the following: Bacteroides, Eubacterium, Fusobacterium, Propionibacterium, Lactobacillus, Ruminococcus, Escherichia coli, Gemmiger, Clostridium, Desulfomonas, Peptostreptococcus, Bifidobacterium, Collinsella, Coprococcus, Dorea, and Ruminococcus.

In an aspect, a therapeutic composition is combined with other adjuvants such as antacids to dampen bacterial inactivation in the stomach. (e.g., Mylanta, Mucaine, Gastrogel). In another aspect, acid secretion in the stomach could also be pharmacologically suppressed using H2-antagonists or proton pump inhibitors. An example H2-antagonist is ranitidine. An example proton pump inhibitor is omeprazole. In one aspect, an acid suppressant is administered prior to administering, or in co-administration with, a therapeutic composition.

In an aspect, a therapeutic composition is administered in the form of: an enema composition which can be reconstituted with an appropriate diluent; enteric-coated capsules; enteric-coated microcapsules; acid-resistant tablet; acid-resistant capsules; acid-resistant microcapsules; powder for reconstitution with an appropriate diluent for naso-enteric infusion or colonoscopic infusion; powder for reconstitution with appropriate diluent, flavoring and gastric acid suppression agent for oral ingestion; powder for reconstitution with food or drink; or food or food supplement comprising enteric-coated and/or acid-resistant microcapsules of the composition, powder, jelly, or liquid.

In an aspect, a treatment method effects a cure, reduction of the symptoms, or a percentage reduction of symptoms of a disorder (e.g., IBD such as ulcerative colitis or Crohn's disease). The change of flora is preferably as “near-complete” as possible and in one embodiment the flora is replaced by viable organisms which will crowd out any remaining, original flora. Typically the change in enteric flora comprises introduction of an array of predetermined flora into the gastro-intestinal system, and thus in a preferred form the method of treatment comprises substantially or completely displacing pathogenic enteric flora in patients requiring such treatment.

In another aspect, a therapeutic composition can be provided together with a pharmaceutically acceptable carrier. As used herein, a “pharmaceutically acceptable carrier” refers to a non-toxic solvent, dispersant, excipient, adjuvant, or other material which is mixed with a live bacterium in order to permit the formation of a pharmaceutical composition, e.g., a dosage form capable of administration to the patient. A pharmaceutically acceptable carrier can be liquid (e.g., saline), gel or solid form of diluents, adjuvant, excipients or an acid resistant encapsulated ingredient. Suitable diluents and excipients include pharmaceutical grades of physiological saline, dextrose, glycerol, mannitol, lactose, starch, magnesium stearate, sodium saccharin, cellulose, magnesium carbonate, and the like, and combinations thereof. In another aspect, a therapeutic composition may contain auxiliary substances such as wetting or emulsifying agents, stabilizing or pH buffering agents. In an aspect, a therapeutic composition contains about 1%-5%, 5%-10%, 10%-15%, 15-20%, 20%-25%, 25-30%, 30-35%, 40-45%, 50%-55%, 1%-95%, 2%-95%, 5%-95%, 10%-95%, 15%-95%, 20%-95%, 25%-95%, 30%-95%, 35%-95%, 40%-95%, 45%-95%, 50%-95%, 55%-95%, 60%-95%, 65%-95%, 70%-95%, 45%-95%, 80%-95%, or 85%-95% of active ingredient. In an aspect, a therapeutic composition contains about 2%-70%, 5%-60%, 10%-50%, 15%-40%, 20%-30%, 25%-60%, 30%-60%, or 35%-60% of active ingredient.

In an aspect, a therapeutic composition can be incorporated into tablets, drenches, boluses, capsules or premixes. Formulation of these active ingredients into such dosage forms can be accomplished by means of methods well known in the pharmaceutical formulation arts. See, e.g., U.S. Pat. No. 4,394,377. Filling gelatin capsules with any desired form of the active ingredients readily produces capsules. If desired, these materials can be diluted with an inert powdered diluent, such as sugar, starch, powdered milk, purified crystalline cellulose, or the like to increase the volume for convenience of filling capsules.

In an aspect, conventional formulation processes can be used to prepare tablets containing a therapeutic composition. In addition to the active ingredients, tablets may contain a base, a disintegrator, an absorbent, a binder, and a lubricant Typical bases include lactose, sugar, sodium chloride, starch and mannitol. Starch is also a good disintegrator as is alginic acid. Surface-active agents such as sodium lauryl sulfate and dioctyl sodium sulphosuccinate are also sometimes used. Commonly used absorbents include starch and lactose. Magnesium carbonate is also useful for oily substances. As a binder there can be used, for example, gelatin, gums, starch, dextrin, polyvinyl pyrrolidone and various cellulose derivatives. Among the commonly used lubricants are magnesium stearate, talc, paraffin wax, various metallic soaps, and polyethylene glycol.

In an aspect, for preparing solid compositions such as tablets, an active ingredient is mixed with a pharmaceutical carrier, e.g., conventional tableting ingredients such as corn starch, lactose, sucrose, sorbitol, talc, stearic acid, magnesium stearate, dicalcium phosphate or gums, or other pharmaceutical diluents, e.g. water, to form a solid preformulation composition containing a homogeneous mixture of a composition of the present disclosure. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation composition is then subdivided into unit dosage forms of the type described above containing a desired amount of an active ingredient (e.g., at least about 10⁵, 10⁶, 10⁷, 10⁸, 10⁹, 10¹⁰, 10¹¹, 10¹², or 10¹³ cfu or total cell count). A therapeutic composition used herein can be flavored.

In an aspect, a therapeutic composition can be a tablet or a pill. In one aspect, a tablet or a pill can be coated or otherwise compounded to provide a dosage form affording the advantage of prolonged action. For example, a tablet or pill can comprise an inner dosage and an outer dosage component, the latter being in the form of an envelope over the former. The two components can be separated by an enteric layer which serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

In an aspect, a therapeutic composition is formulated as a delayed or gradual enteric release form. In an aspect, a delayed or gradual enteric release formulation comprises the use of cellulose acetate, polyethylene glycerol, or both. In an aspect, a delayed or gradual enteric release formulation comprises the use of a hydroxypropylmethylcellulose (HPMC), a microcrystalline cellulose (MCC), magnesium stearate, or a combination thereof. In an aspect, a delayed or gradual enteric release formulation comprises the use of a poly(meth)acrylate, a methacrylic acid copolymer B, a methyl methacrylate, a methacrylic acid ester, a polyvinylpyrrolidone (PVP), a PVP-K90, or a combination thereof. In an aspect, a delayed or gradual enteric release formulation comprises the use of a solid inner layer sandwiched between two outer layers; wherein the solid inner layer comprises the pharmaceutical composition and another component selected from the group consisting of a disintegrant, an exploding agent, an effervescent or any combination thereof; wherein the outer layer comprises a substantially water soluble, a crystalline polymer, or both. In an aspect, a delayed or gradual enteric release formulation comprises the use of a non-swellable diffusion matrix.

In another aspect, a delayed or gradual enteric release formulation comprises the use of a bilayer tablet or capsule which comprises a first layer comprising a polyalkylene oxide, a polyvinylpyrrolidone, a lubricant, or a mixture thereof, and a second osmotic push layer comprising polyethylene oxide, carboxy-methylcellulose, or both. In an aspect, a delayed or gradual enteric release formulation comprises the use of a release-retarding matrix material selected from the group consisting of an acrylic polymer, a cellulose, a wax, a fatty acid, shellac, zein, hydrogenated vegetable oil, hydrogenated castor oil, polyvinylpyrrolidine, a vinyl acetate copolymer, a vinyl alcohol copolymer, polyethylene oxide, an acrylic acid and methacrylic acid copolymer, a methyl methacrylate copolymer, an ethoxyethyl methacrylate polymer, a cyanoethyl methacrylate polymer, an aminoalkyl methacrylate copolymer, a poly(acrylic acid), a poly(methacrylic acid), a methacrylic acid alkylamide copolymer, a poly(methyl methacrylate), a poly(methacrylic acid anhydride), a methyl methacrylate polymer, a polymethacrylate, a poly(methyl methacrylate) copolymer, a polyacrylamide, an aminoalkyl methacrylate copolymer, a glycidyl methacrylate copolymer, a methyl cellulose, an ethylcellulose, a carboxymethylcellulose, a hydroxypropylmethylcellulose, a hydroxymethyl cellulose, a hydroxyethyl cellulose, a hydroxypropyl cellulose, a crosslinked sodium carboxymethylcellulose, a crosslinked hydroxypropylcellulose, a natural wax, a synthetic wax, a fatty alcohol, a fatty acid, a fatty acid ester, a fatty acid glyceride, a hydrogenated fat, a hydrocarbon wax, stearic acid, stearyl alcohol, beeswax, glycowax, castor wax, camauba wax, a polylactic acid, polyglycolic acid, a co-polymer of lactic and glycolic acid, carboxymethyl starch, potassium methacrylate/divinylbenzene copolymer, crosslinked polyvinylpyrrolidone, poly inylalcohols, polyvinylalcohol copolymers, polyethylene glycols, non-crosslinked polyvinylpyrrolidone, polyvinylacetates, polyvinylacetate copolymers, or any combination thereof. In an aspect, a delayed or gradual enteric release formulation comprises the use of a microenvironment pH modifier.

In an aspect, a therapeutic composition can be a drench. In one aspect, a drench is prepared by choosing a saline-suspended form of a therapeutic composition. A water-soluble form of one ingredient can be used in conjunction with a water-insoluble form of the other by preparing a suspension of one with an aqueous solution of the other. Water-insoluble forms of either active ingredient may be prepared as a suspension or in some physiologically acceptable solvent such as polyethylene glycol. Suspensions of water-insoluble forms of either active ingredient can be prepared in oils such as peanut, corn, sesame oil or the like; in a glycol such as propylene glycol or a polyethylene glycol; or in water depending on the solubility of a particular active ingredient. Suitable physiologically acceptable adjuvants may be necessary in order to keep the active ingredients suspended. Adjuvants can include and be chosen from among the thickeners, such as carboxymethylcellulose, polyvinyl pyrrolidone, gelatin and the alginates. Surfactants generally will serve to suspend the active ingredients, particularly the fat-soluble propionate-enhancing compounds. Most useful for making suspensions in liquid nonsolvents are alkylphenol polyethylene oxide adducts, naphthalenesulfonates, alkylbenzene-sulfonates, and the polyoxyethylene sorbitan esters. In addition many substances, which affect the hydrophilicity, density and surface tension of the liquid, can assist in making suspensions in individual cases. For example, silicone anti-foams, glycols, sorbitol, and sugars can be useful suspending agents.

In an aspect, a therapeutic composition comprises non-pathogenic spores of one or more, two or more, three or more, or four or more Clostridium species selected from the group consisting of Clostridium absonum, Clostridium argentinense, Clostridium baratii, Clostridium botulinum, Clostridium cadaveris, Clostridium carnis, Clostridium celatum, Clostridium chauvoei, Clostridium clostridioforme, Clostridium cochlearium, Clostridium fallax, Clostridium felsineum, Clostridium ghonii, Clostridium glycolicum, Clostridium haemolyticum, Clostridium hastiforme, Clostridium histolyticum, Clostridium indolis, Clostridium irregulare, Clostridium limosum, Clostridium malenominatum, Clostridium novyi, Clostridium oroticum, Clostridium paraputrificum, Clostridium perfringens, Clostridium pihforme, Clostridium putrefaciens, Clostridium putrificum, Clostridium sardiniense, Clostridium sartagoforme, Clostridium scindens, Clostridium septicum, Clostridium sordellii, Clostridium sphenoides, Clostridium spiroforme, Clostridium sporogenes, Clostridium subterminale, Clostridium symbiosum, Clostridium tertium, Clostridium tetani, Clostridium welchii, and Clostridium villosum.

In an aspect, a therapeutic composition comprises purified, isolated, or cultured viable non-pathogenic Clostridium and a plurality of purified, isolated, or cultured viable non-pathogenic microorganisms from one or more genera selected from the group consisting of Collinsella, Coprococcus, Dorea, Eubacterium, and Ruminococcus. In another aspect, a therapeutic composition comprises a plurality of purified, isolated, or cultured viable non-pathogenic microorganisms from one or more genera selected from the group consisting of Clostridium, Collinsella, Coprococcus, Dorea, Eubacterium, and Ruminococcus.

In an aspect, a therapeutic composition comprises two or more genera selected from the group consisting of Collinsella, Coprococcus, Dorea, Eubacterium, and Ruminococcus. In another aspect, a therapeutic composition comprises two or more genera selected from the group consisting of Coprococcus, Dorea, Eubacterium, and Ruminococcus. In a further aspect, a therapeutic composition comprises one or more, two or more, three or more, four or more, or five or more species selected from the group consisting of Coprococcus catus, Coprococcus comes, Dorea longicatena, Eubacterium eligens, Eubacterium hadrum, Eubacterium hallii, Eubacterium rectale, and Ruminococcus torques.

In one aspect, a pharmaceutical composition is in an anaerobic package or container. In another aspect, a pharmaceutical composition further comprises an oxygen scavenger. In one aspect, a container can be made oxygen free by e.g., incorporating into the container a built in or clipped-on oxygen-scavenging mechanism, e.g., oxygen scavenging pellets as described e.g., in U.S. Pat. No. 7,541,091. In another aspect, the container itself is made of an oxygen scavenging material, e.g., oxygen scavenging iron, e.g., as described by O2BLOCK™, or equivalents, which uses a purified and modified layered clay as a performance-enhancing carrier of oxygen-scavenging iron; the active iron is dispersed directly in the polymer. In one aspect, oxygen-scavenging polymers are used to make the container itself or to coat the container, or as pellets to be added; e.g., as described in U.S. Pat. App. Pub. 20110045222, describing polymer blends having one or more unsaturated olefinic homopolymers or copolymers; one or more polyamide homopolymers or copolymers; one or more polyethylene terephthalate homopolymers or copolymers; that exhibit oxygen-scavenging activity. In one aspect, oxygen-scavenging polymers are used to make the container itself or to coat the container, or as pellets to be added; e.g., as described in U.S. Pat. App. Pub. 20110008554, describing compositions comprising a polyester, a copolyester ether and an oxidation catalyst, wherein the copolyester ether comprises a polyether segment comprising poly(tetramethylene-co-alkylene ether). In one aspect, oxygen-scavenging polymers are used to make the container itself or to coat the container, or as pellets to be added; e.g., as described in U.S. Pat. App. Pub. 201000255231, describing a dispersed iron/salt particle in a polymer matrix, and an oxygen scavenging film with oxygen scavenging particulates.

In one aspect, of the pharmaceutical compositions provided herein, the pharmaceutical compositions are manufactured, labelled or formulated for human or animal use, and optionally the animal use is for a veterinary use.

In one aspect, the pharmaceutical compositions provided herein are further processed or manufactured or formulated as a liquid, a suspension, a cream, a gel, a geltab, a semisolid, a tablet, a sachet, a lozenge or a capsule, or as an enteral formulation, or re-formulated for final delivery as a liquid, a suspension, a gel, a geltab, a semisolid, a tablet, a sachet, a lozenge or a capsule, or as an enteral formulation.

In one aspect, provided herein are delivery vehicles, products of manufacture, containers or devices comprising a pharmaceutical composition as provided herein, optionally formulated for or calibrated for repeat or multiple implantations, administration, delivery or infusions.

In one aspect, provided herein are delivery vehicles, products of manufacture, containers or devices, further comprising one or more of: an additive, a media, a defoaming agent, a surfactant agent, a lubricant, an acid neutralizer, a marker, a cell marker, a drug, an antibiotic, a contrast agent, a dispersal agent, a buffer or a buffering agent, a sweetening agent, a debittering agent, a flavoring agent, a pH stabilizer, an acidifying agent, a preservative, a desweetening agent, coloring agent, at least one vitamin, mineral and/or dietary supplement, or a prebiotic nutrient.

In one aspect, provided herein are methods for making a pharmaceutical composition comprising a lyophilized, cryodesiccated, freeze-dried or dehydrated bacterial flora, comprising:
(a) providing a composition, isolate or preparation comprising, or consisting essentially of, or consisting of:
   an entire (or substantially entire) fecal microbiota, optionally isolated and/or stored in a partially, substantially or completely anaerobic environment,
   a treated or untreated fecal flora sample,
   a complete or partial fecal flora sample, optionally isolated and/or stored in a partially, substantially or completely anaerobic environment,
   a fecal flora substantially or completely purified of non-fecal floral fecal material, wherein optionally the fecal flora is separated from a rough particulate matter in a fecal sample by: homogenizing, centrifuging and/or filtering a rough particulate matter or a non-floral matter of the fecal material, or by plasmapheresis, centrifugation, celltrifuge, column chromatography or by immunoprecipitation, and optionally the substantially or completely purified fecal flora has no greater than about 0.05%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, or 10% weight non-living material/weight biological material,
   a partially, substantially or completely isolated or purified fecal flora or fecal flora filtrate, wherein optionally the purification process comprises filtering a fecal sample with a filter medium, wherein optionally the filter medium includes at least one sieve size of no greater than about 2.0 mm, 1.0 mm, 0.5 mm, 0.25 mm, 0.212 mm, 0.180 mm, 0.150 mm, 0.125 mm, 0.106 mm, 0.090 mm, 0.075 mm, 0.063 mm, 0.053 mm, 0.045 mm, 0.038 mm, 0.032 mm, 0.025 mm, 0.020 mm, or 0.01 mm, or a sieve size of 2.0 mm, 1.0 mm, 0.5 mm, 0.25 mm, 0.212 mm, 0.180 mm, 0.150 mm, 0.125 mm, 0.106 mm, 0.090 mm, 0.075 mm, 0.063 mm, 0.053 mm, 0.045 mm, 0.038 mm, 0.032 mm, 0.025 mm, 0.020 mm, 0.01 mm, or 0.2 mm, to result in or to generate a filtrate,
   a disease screened fresh homologous feces, optionally substantially or completely purified of non-fecal floral fecal material, or optionally isolated and/or stored in a partially, substantially or completely anaerobic environment, and optionally the fecal flora is initially derived from an individual screened or tested for a disease or an infection, and/or the fecal flora is initially derived from an individual screened to have a normal, healthy or wild type population of fecal flora,
   a reconstituted feces, optionally reconstituted using cultured viable non-pathogenic or attenuated microorganisms,
   a synthetic fecal composition of predetermined flora,
   a synthetic or reconstituted fecal composition comprising a preparation of viable flora in proportional content that resembles a normal healthy human fecal flora, which does not include antibiotic resistant populations,
   a composition comprising viable, non-pathogenic colonic bacterial cells selected from the group consisting of a Clostridia, a Collinsella, a Bacteroides, a Fusobacteria, a Propionibacteria, a Lactobacilli, an anaerobic cocci, a Ruminococcus, an E. coli, a Gemmiger, a Desulfomonas, a Peptostreptococcus, a Bifidobacteria and any combination thereof;
   a composition comprising viable, non-pathogenic colonic bacterial components of fecal flora, wherein the bacterial components comprise Clostridium bifermentans, Clostridium innocuum, Clostridium butyricum, Escherichia coli, Bacteroides and Peptostreptococcus productus,
   a composition comprising viable, non-pathogenic colonic bacterial components of fecal flora, wherein the bacterial components comprise a Bacteroides, an Escherichia coli, and a non-pathogenic Clostridia, wherein optionally the non-pathogenic Clostridia comprise a Clostridium innocuum, a Clostridium bifermentans and a Clostridium ramosum,
   a composition comprising a plurality of viable non-pathogenic Clostridia and a plurality of viable non-pathogenic Collinsella, and optionally with no viable Lactobacilli, Bifidobacteria or Eubacteria, and optionally with no viable Bacteroides, Fusobacteria, Propionibacteria, Lactobacilli, anaerobic cocci, Ruminococcus, E. coli, Gemmiger, Desulfomonas, Peptostreptococcus or Bifidobacteria,
   a composition comprising a plurality of viable non-pathogenic Clostridia, wherein optionally the plurality of viable non-pathogenic Clostridia comprise non-pathogenic Clostridia spores,
   a composition comprising a plurality of viable non-pathogenic Clostridia and a plurality of viable non-pathogenic Collinsella, wherein optionally the plurality of viable non-pathogenic Clostridia comprise non-pathogenic Clostridia spores and/or the plurality of viable non-pathogenic Clostridia comprise non-pathogenic Collinsella spores, wherein optionally the plurality of viable non-pathogenic Clostridia and are from a first pure culture and the plurality of viable non-pathogenic Collinsella cells from a second pure culture, and/or
   a composition comprising viable non-pathogenic Clostridia spores, a viable non-pathogenic Bacteroides, and a viable non-pathogenic Escherichia coli,
   wherein optionally the composition, isolate or preparation has at least about 10⁶ viable cells/g, 10⁷ viable cells/g, 10⁸ viable cells/g, at least about 10⁹ viable cells/g, 10¹⁰ viable cells/g, 10¹¹ viable cells/g, 10¹² viable cells/g, 10¹³ viable cells/g, 10¹⁴ viable cells/g, or 10¹⁵ viable cells/g, or between about 10⁶ and 10¹⁵, between about 10⁸ and 10¹⁵ viable cells/g, or between about 10¹⁰ and 10¹² viable cells/g;
(b) providing a cryoprotectant and optionally a surfactant or an emulsifier,
wherein optionally the cryoprotectant comprises: a dimethyl sulfoxide (DMSO) or equivalent; a glycerol, a polyethylene glycol (PEG) or equivalent; a polysaccharide; a sugar, or an amino acid,
wherein optionally the amino acid comprises an alanine, a glycine, a proline, or the sugar comprises a mannitol, a sucrose, a glucose, a lactose, a ribose or trehalose, or the polysaccharide comprises a hydroxypropyl-β-cyclodextrin (HPβCD), or the cryoprotectant comprises any combination of different cryoprotectant compounds,
wherein optionally the surfactant or emulsifier comprises a polysorbate (polyoxyethylene sorbitan monolaurate) or a PEG-ylated sorbitan, optionally a Polysorbate 80 (polyoxyethylene (80) sorbitan monolaurate);
(c) homogenizing the composition, isolate or preparation of (a) with a mixture of saline and cryoprotectant, or with a mixture of saline, cryoprotectant and a surfactant or an emulsifier,
wherein optionally the homogenization is about 1:2, 1:3, 1:4 or 1:5 (w/w) with a solution comprising saline,
and optionally the cryoprotectant is present at a concentration of at least 1%, 2%, 3%, 4%, 5%, 6%, 7%, 8%, 9%, or 10% (vol/vol);
(d) lyophilizing, cryodesiccating, freeze-drying or dehydrating the homogenized composition, isolate or preparation mixture of (c),
wherein optionally after the lyophilizing, cryo-desiccating, freeze-drying or dehydrating the final water activity (aw) is less than about 0.1, 0.2, 0.3 or 0.4; and
(e) storing, keeping and/or maintaining the lyophilized, cryo-desiccated, freeze-dried or dehydrated composition, isolate or preparation at ambient temperature, room temperature, approximately room temperature, freezing temperature, or at between about 2° C. to 8° C., or at between about 15° C. to 26° C., or at about 20° C., 21° C., 22° C., 23° C., 24° C., 25° C., 26° C., 27° C. or 28° C.,
   wherein optionally the stored pharmaceutical composition has at least about 10⁸ viable cells/g, 10⁹ viable cells/g, 10¹⁰ viable cells/g, 10¹² viable cells/g, 10¹³ viable cells/g, or 10¹⁴ viable cells/g, or between about 10⁷ and 10¹² viable cells/g, or between about 10⁹ and 10¹¹ viable cells/g.

In one aspect, the pharmaceutical compositions are formulated in a gastric acid resistant capsule.

In one aspect, provided are methods for treating a disorder in a subject in need thereof, the method comprising administering to the subject an amount of the pharmaceutical composition disclosed herein, wherein the disorder is selected from the group consisting of recurrent C. diff infection, autism, constipation predominant functional bowel disease (FBD), pain predominant FBD, upper abdominal FBD, non-ulcer dyspepsia (NUD), gastro-oesophageal reflux, indeterminate colitis, microscopic colitis, pseudomembranous colitis, viral gastroenteritis, Norwalk viral gastroenteritis, rotavirus gastroenteritis, AIDS related gastroenteritis, non-rheumatoid factor positive arthritis, Lyme disease, systemic lupus, idiopathic thrombocytopenic purpura, Sjogren's syndrome, haemolytic uremic syndrome or scleroderma, Gillain-Barre syndrome, Chronic Inflammatory Demyelinating Polyneuropathy, chronic depression, schizophrenia, psychotic disorders, manic depressive illness, Asbergers syndrome, Rett syndrome, attention deficit hyperactivity disorder (ADHD), and attention deficit disorder (ADD), sudden infant death syndrome (SIDS), anorexia nervosa.

In one aspect, provided are methods comprising storing the pharmaceutical composition as provided herein at ambient temperature, room temperature, approximately room temperature, freezing temperature, or at between about 2° C. to 8° C., or at between about 15° C. to 26° C., or at about 20° C., 21° C., 22° C., 23° C., 24° C., 25° C., 26° C., 27° C. or 28° C.; and optionally, at least 50%, at least 55%, at least 60%, at least 65%, at least 70%, at least 75%, at least 80%, at least 85%, at least 90%, or at least 95% microbial viability of the microflora material of the pharmaceutical composition is maintained, or between about 40% and 95% of the microflora material of the pharmaceutical composition is maintained, after about 2, about 4, about 8, about 12, about 20, about 30, about 40, about 50, or about 60 weeks of storage from (after) preparation of the pharmaceutical composition, or after between about 2 to 60 weeks from (after) preparation of the pharmaceutical composition, or after between about 1 to 12 months, or 2 to 24 months, from (after) preparation of the pharmaceutical composition.

In one aspect, provided are methods and compositions facilitating prolonged viability or longer term survival of FMT, e.g., filtered fecal microbiota, at e.g., ambient temperatures, e.g., at room temperatures (including storage, including long term storage at ambient temperatures, e.g., at room temperatures); and while the disclosure is not limited by any particular mechanism of action, this prolonged viability or longer term survival can be achievable through the use of a cryoprotectant and/or a mix of cryoprotectants at various mix compositions, thus, storage at ambient temperature (e.g., prolonged shelf-life in pharmacy or home) may be achieved. In one aspect, the cryoprotectants and associated liquids include trehalose, sucrose, normal saline, mannitol, and polysorbate(s), e.g., a polysorbate 80, in various combinations.

In one aspect, provided are compositions having the ability to isolate, prepare, formulate and/or reduce the volume of the FMT product so as to store it in a delivery system, e.g., as a bottle-top of a drink, e.g., a chocolate drink, as a side compartment of yoghurt, or a two-layered aluminized top of ice-cream tub for kids, e.g., for autism, and methods of preparation of same.

In one aspect, provided are compositions prepared and/or formulated in a powdered form, or equivalent; these formulations can be useful for storage in e.g., a tablet or capsule, or in an ampoule to e.g., crack open and dissolve in a liquid for, e.g., insertion, mixing or injection into e.g. a channel of a colonoscope or a naso-enteric tube, and the like; or as a powder in a bag ready to add e.g., as a solution which can be e.g., infused into an NG tube (or equivalent), or a colonoscope, or a gastroscope for e.g., stoma gastrostomy, or a PEG tube.

In one aspect, provided are freeze dried or lyophilized materials, which can be formulated or manufactured into or as an edible or friable product, e.g., a biscuit-like product, which can be e.g., crushed into a powder to dissolve in a drink or to insert into a tablet or a capsule. In one aspect, provided are FMT-comprising formulations that are orally ingestible, or can be a rectally applied product. In one aspect, provided are FMT-comprising formulations in the form of a dry lozenge or a chewing gum or equivalent. In one aspect, use of all of these formulations, foods, drinks, and products of manufacture are facilitated by the ability to manufacture, ship and store at room temperature, at an ambient temperature, at a freezing temperature, or at between about 2° C. to 8° C., as provided by this disclosure.

In one aspect, provided herein are compositions, e.g., formulations and pharmaceutical preparations, products of manufacture, and containers and delivery vehicles, and devices and delivery materials, comprising treated and/or isolated faecal (fecal) material for faecal floral transplantation. In one aspect, the treated and/or isolated fecal material provided herein comprise faecal floral (e.g., bacteria) transplanted between different individuals, e.g., human to human or between animals. In one aspect, the treated fecal material provided herein is transplanted back into the same individual from which it was collected, e.g., to repopulate a colon after drug treatment (e.g., antibiotic treatment or chemotherapy) or after an orthostatic lavage, e.g., for inducing the purgation (e.g., cleansing) of a gastrointestinal (GI) tract, including a colon.

In one aspect, compositions, e.g., formulations and pharmaceutical preparations, products of manufacture, and containers and delivery vehicles, and devices and delivery materials provided herein are used for the amelioration, stabilization, or treatment of a bowel disease or infection comprising use of a delivery vehicle, formulation, product of manufacture, or container or device provided herein; e.g., as a fecal bacteriotherapy, fecal transfusion, fecal transplant, or human probiotic infusion (HPI). In one aspect, provided herein are methods for using compositions provided herein for e.g., ameliorating, stabilizing, treating or preventing any infection, bowel disease or condition having a bowel dysfunction component, for example, a poisoning, a pseudomembranous colitis, a Clostridium difficile infection, an inflammatory bowel disease (IBD), Crohn's disease, hepatic encephalopathy, enteritis, colitis, irritable bowel syndrome (IBS), fibromyalgia (FM), alopecia areata/totalis, anorexia nervosa, autism, chronic fatigue syndrome (CFS), depression, attention deficit/hyperactivity disorder (ADHD), multiple sclerosis (MS), systemic lupus erythematosus (SLE), travellers' diarrhea, small intestinal bacterial overgrowth, chronic pancreatitis, or a pancreatic insufficiency.

For example, in one aspect, as antibiotics do not eradicate C. difficile and its spore, a delivery vehicle, formulation, product of manufacture, or container or device as provided herein, e.g., comprise treated and/or isolated fecal flora for use to ameliorate, stabilize or eradicate C. difficile (or the pseudo-membranous colitis associated with this infection) when infused into a colon of the infected or ill individual, e.g., a patient or animal. In one aspect, the fecal flora obtained from a donor comprises a part of, substantially all of, or all of the infected or ill recipient's missing or inadequate (e.g., in numbers or function) fecal flora, e.g., bacteria. While the disclosure is not limited by any particular mechanism of action, in some aspects it is the transfer of the equivalent of: a part of, substantially all of, or all of the fecal flora of the infected individual from the donor to the recipient (e.g., from human to human) that ameliorates or eradicates the infection or the pseudo-membranous colitis associated with this infection.

In one aspect, the compositions, e.g., formulations and pharmaceutical preparations, and devices, delivery materials, delivery vehicles, products of manufacture, containers and devices provided herein allow the safe transplantation of fecal flora (e.g., human flora) components to individuals in need thereof, e.g., to infected, sick and dying patients, thus providing a consistently safe yet functioning flora for delivery to a recipient or patient.

In one aspect, provided herein is a reliable method for producing standardized fresh fecal flora which can have a long shelf life. In one aspect, the delivery vehicle, formulation, pharmaceutical preparation, product of manufacture, container or device comprising the fecal flora comprises a substantially or completely oxygen-free environment. In another aspect, nutrients such as “prebiotic nutrients” can be added (e.g., in dry or liquid forms) to a composition provided herein. A prebiotic nutrient can be any ingredient that stimulates the stability, growth and/or activity of the fecal flora, e.g., bacteria; for example, in one aspect, polyols, fructooligosaccharides (FOSs), oligofructoses, inulins, galactooligosaccharides (GOSs), xylooligosaccharides (XOSs), polydextroses, monosaccharides such as tagatose, and/or mannooligosaccharides are used as prebiotics to practice this disclosure. In one aspect, the prebiotics are added to prevent “shock” to the fecal flora subsequent to their isolation or purification, freezing, freeze-drying, spray-drying, reconstitution in solution and the like.

In one aspect, components of the compositions, e.g., delivery vehicles, formulations and pharmaceutical preparations, products of manufacture, or containers or devices, provided herein comprise an entire (or substantially entire) microbiota, or a Bacteroides and/or Firmicutes in large numbers (e.g., a larger proportion of Bacteroides and/or Firmicutes is present that is normally found in situ), e.g., to be able to ameliorate and/or eradicate a C. difficile infection and/or the pseudo-membranous colitis associated with this infection. In one aspect, the compositions, e.g., delivery vehicles, formulations and pharmaceutical preparations, products of manufacture, or containers or devices, provided herein can be available (e.g., formulated and/or dosaged for) for recurrent use in individuals, e.g., in patients or animals, with the more difficult to treat conditions such as colitis (e.g., the pseudo-membranous colitis of a C. difficile infection) and constipation.

In one aspect, components of the compositions e.g., delivery vehicles, formulations and pharmaceutical preparations, products of manufacture, or containers or devices, provided herein comprise a selection of bacterial species e.g. Bacteroides, Firmicutes, Bacillus thuringiensis (a bacterium capable of producing peptide antibiotics for C. difficile). The bacterial species may be separated by celltrifugation or plasmapheresis.

In one aspect, the selection of bacterial species e.g. Bacteroides, Firmicutes, Bacillus thuringiensis may be added to components of the compositions, e.g., delivery vehicles, formulations and pharmaceutical preparations, products of manufacture, or containers or devices as fortification of concentrations comprising the bacterial species to contain wild types of bacteria.

In one aspect, compositions provided herein can be re-formulated as fecal slurries, saline or buffered suspensions (e.g., for an enema, suspended in a buffer or a saline), in a drink (e.g., a milk, yoghurt, a shake, a flavoured drink or equivalent) for oral delivery, and the like.

In one aspect, compositions provided herein can be formulated or re-formulated as an enema product, a spray dried product, reconstituted enema, a small capsule product, a small capsule product suitable for administration to children, a bulb syringe, a bulb syringe suitable for a home enema with a saline addition, a powder product, a powder product in oxygen deprived sachets, a powder product in oxygen deprived sachets that can be added to, for example, a bulb syringe or enema, or a spray dried product in a device that can be attached to a container with an appropriate carrier medium such as yoghurt or milk and that can be directly incorporated and given as a dosing for example for children.

In one aspect, compositions provided herein can be delivered directly in a carrier medium via a screw-top lid wherein the fecal material is suspended in the lid and released on twisting the lid straight into the carrier medium.

In one aspect, provided herein include fecal slurries formulated for insertion/administration into the bowel, e.g., via an enema suspended in saline or a buffer, orally in a drink (e.g., a milk, yoghurt, a flavoured drink and the like), via a small bowel infusion via a nasoduodenal tube, via a gastrostomy, or by using a colonoscope. In some aspect, there may be advantages delivering via a colonoscope to infuse as proximally as possible, and to detect any colonic pathology.

In one aspect, methods, fecal flora used in compositions provided herein are initially derived (entirely or in part) from an individual screened or tested for a disease or infection, and/or the fecal flora is initially derived from an individual screened to have a normal, healthy or normal, representative “wild type” population of fecal flora; e.g., a normal complement of a Bacteroides and/or Firmicutes, and/or other fecal flora such as Bacillus Thuringiensis. In one aspect, depending on a deficiency of a floral (e.g., bacterial) specie or species in a donor fecal material, or to achieve a desired effect, one or more additional (or “supplemental”) species, e.g., Bacteroides, Firmicutes and/or Bacillus Thuringiensis species, is added to (or is administered with) the delivered product either initially when the product is made, or at the time of delivery, e.g., the additional species is/are mixed in before application to the individual (e.g., patient or animal), e.g., when a powder, lyophilate, or freeze-dried composition is reconstituted for delivery; or the one or more additional (or “supplemental”) species can be co-administered. These additional floral species can be directly isolated or purified from a donor, or can be expanded (cultured) for a time in vitro before addition, or can come from (be derived from) a pure culture, e.g., from an ATTC stock. For example, in some applications, e.g., to achieve a desired effect or therapeutic outcome, a delivery of an enhanced amount of one or more fecal flora (e.g., bacterial) species is used, e.g., the delivered product (e.g., an entire (or substantially entire) microbiota, or a composition comprising a complete or partial fecal flora, or a partially, substantially or completely isolated or purified fecal flora) is enhanced with (is “spiked” with”) one or more additional (or “supplemental”) species, e.g., Bacteroides, Firmicutes and/or Bacillus Thuringiensis species, which can be directly isolated from a donor, or can come from a pure culture, and the like.

In some aspects, selection of the donor is of crucial importance, e.g., to avoid infecting the recipient with a separate infection or disease. In one aspect, the donor is tested (screened) at least for e.g., retrovirus (e.g., human immunodeficiency virus, HIV); hepatitis A, B, and/or C; cytomegalovirus; Epstein-Barr virus, detectable parasites and/or bacterial pathogens, depending on the specie of the donor and recipient, e.g., human or animal.

In one aspect, provided herein is a process for preparing fecal flora (e.g., an entire (or substantially entire) microbiota) for transplantation, first comprising a collection from one or more healthy (e.g., screened) donor(s). In one aspect, a fresh stool is transported via a stool collection device, which can provide or comprises a suitably oxygen free (or substantially oxygen free) appropriate container. In one aspect, the container can be made oxygen free by e.g., incorporating into the container a built in or clipped-on oxygen-scavenging mechanism, e.g., oxygen scavenging pellets as described e.g., in U.S. Pat. No. 7,541,091. In another aspect, the container itself is made of an oxygen scavenging material, e.g., oxygen scavenging iron, e.g., as described by O2BLOCK™, or equivalents, which uses a purified and modified layered clay as a performance-enhancing carrier of oxygen-scavenging iron; the active iron is dispersed directly in the polymer. In one aspect, oxygen-scavenging polymers are used to make the container itself or to coat the container, or as pellets to be added; e.g., as described in U.S. Pat. App. Pub. 20110045222, describing polymer blends having one or more unsaturated olefinic homopolymers or copolymers; one or more polyamide homopolymers or copolymers; one or more polyethylene terephthalate homopolymers or copolymers; that exhibit oxygen-scavenging activity. In one aspect, oxygen-scavenging polymers are used to make the container itself or to coat the container, or as pellets to be added; e.g., as described in U.S. Pat. App. Pub. 20110008554, describing compositions comprising a polyester, a copolyester ether and an oxidation catalyst, wherein the copolyester ether comprises a polyether segment comprising poly(tetramethylene-co-alkylene ether). In one aspect, oxygen-scavenging polymers are used to make the container itself or to coat the container, or as pellets to be added; e.g., as described in U.S. Pat. App. Pub. 201000255231, describing a dispersed iron/salt particle in a polymer matrix, and an oxygen scavenging film with oxygen scavenging particulates.

Alternatively, in addition to or in place of the oxygen-scavenging mechanism, the air in the container can be replaced (completely or substantially) with nitrogen and/or other inert non-reactive gas or gases. In one aspect, the container simulates (creates) partially, substantially or completely an anaerobic environment.

In one aspect, the stool (e.g., fecal sample) is held in an aesthetically acceptable container that will not leak nor smell yet maintain an anaerobic environment. In one aspect, the container is sterile before receiving the fecal flora.

In one aspect, the compositions provided herein are maintained at room temperature during most or all of its preparation, transportation and/or storage at e.g., a “stool bank” or at the site where the transplantation will take place. For example, once delivered to a “processing stool bank” it is stored in at ambient temperature, e.g., room temperature. In one aspect, stabilizing agents such as glycerol are added to the harvested and/or stored material.

In one aspect, the stool is tested for various pathogens, as noted above. In one aspect, once cleared of infective agents, it is homogenized and filtered to remove large particles of matter. In one aspect, it is subdivided into desired volumes, e.g., which can be between 5 cc and 3 or more liters. For example, in one aspect, a container comprises a 50 gram (g) stool, which can be held in an appropriate oxygen resistant plastic, e.g., a metallized polyethylene terephthalate polyester film, or a metallized MYLAR™.

In one aspect, the FMT material is subject to homogenization.

In one aspect, compositions provided herein are placed into a container, e.g., a bag, that can be attached to a nasogastric or naso-duodenal tube to allow the contents to be infused e.g., into either a stomach, duodenum or the distal jejunum. Alternatively it can be kept in a container, e.g., a bag, which can be attached to an enema tip to be given as an enema.

In one aspect, to separate the non-bacterial components and produce a product that can be lyophilized and have a long shelf life, the stool can be homogenized and filtered from rough particulate matter. In one aspect, the microscopic fiber/nonliving matter is then separated from the bacteria. Several methods can be used, including e.g., recurrent filtration with filter sizes, e.g., coming down to the size of the bacterium.

In one aspect, different filters are used to isolate bacterial spp., or a technique as used by Williams in WO 2011/033310A1, which uses a crude technique of filtration with a gauze.

In one aspect, a filtration procedure for filtering whole stool is suitably used to reach the highest concentration of almost 100% bacteria. In one aspect, the filtering procedure is a two-step procedure suitably using glass fibre depth filters for initial clarification. In one aspect, the stool is filtered under positive pressure. In one aspect, this would be using a combination or sandwich configuration with a 30 micron PVDF filter. In one aspect, this sandwich procedure will be filtering the product under positive pressure. Later, membrane concentration can, in one aspect, be used as another step to reduce the volume of the filtrate. In one aspect, this can be done prior to freeze drying or spray drying under nitrogen cover.

Alternative membranes that can be used for filtration include, but not limited to, nylon filters, cellulose nitrate filters, polyethersulfone (PES) filters, polytetrafluorethylene (PTFE) filters, TEFLON™ filters, mixed cellulose Ester filters, polycarbonate filters, polypropylene filters, Polyvinylchloride (PVC) filters or quartz filters. Various combinations of these can be used to achieve a high purity of bacteria with solids and liquid removed ready for freezing, spray-drying or lyophilisation.

For freeze-drying, in one aspect, bacteria are held in a liquid that will prevent bursting of cells on thawing. This can include various stabilizers, e.g., glycerol and appropriate buffers, and/or ethylene glycol. In one aspect, cryo-protectance uses final concentrations of stabilizer(s) of between about 10% to 80%, 20% to 70%, 30% to 60%, or 40% to 50%, depending on the stabilizer(s) used; in one aspect, this helps stabilize proteins by preventing formation of ice crystals that would otherwise destroy protein structures.

In one aspect, the methods and compositions of the disclosure comprise use of one cryoprotectant or a mixture of cryoprotectants, e.g., comprising: a dimethyl sulfoxide (DMSO) or equivalent; a glycerol, a polyethylene glycol (PEG) or equivalent; a polysaccharide; a sugar, or an amino acid, wherein the amino acid can comprise an alanine, a glycine, a proline, or the sugar can comprise a mannitol, a sucrose, a glucose, a lactose, a ribose or trehalose, or the polysaccharide can comprise a hydroxypropyl-β-cyclodextrin (HPβCD), or the cryoprotectant can comprise any combination of different cryoprotectant compounds. In one aspect, these cryoprotectants, e.g., trehalose, also function as a component upon reconstitution or as an additional agent prior to spray-drying or freeze-drying.

In one aspect, pharmaceutical compositions provided herein comprise lyophilized, cryodesiccated, freeze-dried or dehydrated microflora material from a formulation comprising one or more, two or more, three or more, four or more additives selected from the group consisting of trehalose, mannitol, sucrose, NaCl, and polysorbate 80, wherein the two or more components are effective in reducing or minimizing microbial viability loss in the microflora material. Used herein, additives include, but are not limited to, cryoprotectants, surfactants, and emulsifiers.

In one aspect, stabilizers that help reduce destruction of living bacteria include skim milk, erythritol, arabitol, sorbitol, glucose, fructose and other polyols. Polymers such as dextran and polyethylene glycol can also be used to stabilize the fecal bacterial cells.

In one aspect, an entire (or substantially entire) microbiota, or an isolated and/or treated (e.g., purified or isolated) fecal material and/or flora, is lyophilized or freeze dried, and the product is stored at ambient temperatures (e.g., room temperature), at a freezing temperature, or at between about 2° C. to 8° C. In one aspect, freeze-drying allows the majority of cells to remain viable, and produces a powdered form of the product that can be gently pulverized into a powder. The powder, or lyophilized or freeze-dried flora or isolate, then can be encapsulated into a carrier, e.g., a tablet, geltab, pill or capsule, e.g., an enteric-coated capsule, or placed into oil-filled capsules for ingestion. Alternatively, the freeze-dried or lyophilized product, or powder, can be reconstituted at ambient temperatures before delivery to an individual in e.g., a fluid, e.g., a sterile fluid, such as saline, a buffer or a media such as a fluid-glucose-cellobiose agar (RGCA) media.

In one aspect, an entire (or substantially entire) microbiota, or an isolated and/or treated (e.g., purified or isolated) fecal material and/or flora also can be spray-dried or foam-dried.

In one aspect, the entire (or substantially entire) microbiota, or isolated and/or treated fecal material and/or flora, is supplemented with wild type bacteria which has been derived from normal animal (e.g., human) flora and/or recombinantly treated bacteria, e.g., recombinant microorganisms that can synthesize a protein, small molecule or carbohydrate that has a self-protective or ameliorative effect; or recombinant microorganisms that can self-destruct when provided with an appropriate signal, e.g., a chemical delivered by ingestion.

In some aspects, pharmaceutical compositions provided herein include at least 2 different phyla of gut, colon or intestinal bacteria extracted or prepared from the gut, colon or intestine, and a cryoprotectant, wherein the phyla include a Bacteroidetes, a Firmicutes, a Proteobacteria a Tenericutes phylum, or a combination thereof, wherein optionally the phyla are chosen from Bacteroidetes, Firmicutes, Proteobacteria, Tenericutes, or a combination thereof, wherein the compositions, upon reconstitution with water, include no greater than about 0.05%, 0.1%, 0.2%, 0.3%, 0.4%, 0.5%, 0.6%, 0.7%, 0.8%, 0.9% 1%, 2%, 3%, 4%, 5%, 6%, 7%, 8%, 9%, or 10% weight non-living material/weight biological material, wherein the biological material includes human gut, colon or intestinal fecal microbes, and optionally the biological material includes human gut, colon or intestinal bacteria, and wherein optionally the compositions include a pharmaceutically acceptable carrier, and optionally the composition is a formulation for oral administration.

In some aspects, pharmaceutical compositions provided herein include an extract of human feces and a cryoprotectant, wherein the composition, upon reconstitution with water, is substantially odorless, wherein the composition includes biological material, and optionally wherein the biological material includes microbes, and wherein optionally the composition includes a pharmaceutically acceptable carrier, and optionally the composition is a formulation for oral administration.

In one aspect, the microflora material of a pharmaceutical composition provided herein comprises predominantly spores. In some aspects, at least 50%, 60%, 70%, 80%, 90%, 95%, 97%, 99% of the microbes in the microflora material are in a spore form. “Spore” refers to a microbial entity, which is in a dormant, non-vegetative and non-reproductive stage. Spores are generally resistant to environmental stress including, but not limited to radiation, desiccation, enzymatic treatment, temperature variation, nutrient deprivation, and chemical disinfectants. A collection of spores may be purified from a fecal sample, e.g. via ethanol or heat treatment or other known methods in the art. Alternatively, a collection of spores may be derived through culture methods starting from isolated spore former species or from a mixture of such species, either in vegetative or spore form.

In some aspects, pharmaceutical compositions provided herein comprise non-pathogenic Clostridia spores. In other aspects, pharmaceutical compositions also comprises viable non-pathogenic Collinsella. In some aspects, pharmaceutical compositions further comprise viable non-pathogenic organisms from at least one of the groups consisting of Bacteroides, Fusobacteria, Propionibacteria, Lactobacilli, anaerobic cocci, Ruminococcus, E. coli, Gemmiger, Desulfomonas, Peptostreptococcus, and Bifidobacteria. In further aspects, pharmaceutical compositions further comprises one or more viable non-pathogenic microorganisms selected from the group consisting of a Bacteroides fragilis ss. Vulgatus, Collinsella aerofaciens, Bacteroides fragilis ss. Thetaiotaomicron, Peptostreptococcus productus II, Parabacteroides distasonis Fusobacterium prausnitzii, Coprococcus eutactus, Collinsella aerofaciens III, Peptostreptococcus productus I, Ruminococcus bromii, Bifdobacterium adolescentis, Gemmiger formicilis, Bifidobacterium longum, Eubacterium siraeum, Ruminococcus torques, Eubacterium rectale, Eubacterium eligens, Bacteroides eggerthii, Clostridium leptum, Bacteroides fragilis ss. A, Eubacterium biforme, Bifidobacterium infantis, Eubacterium rectale III-F, Coprococcus comes, Pseudoflavonifractor capillosus, Ruminococcus albus, Dorea formicigenerans, Eubacterium halii, Eubacterium ventriosum I, Fusobacterium russi, Ruminococcus obeum, Eubacterium rectale, Clostridium ramosum, Lactobacillus leichmannii, Ruminococcus callidus, Butyrivibrio crossotus, Acidaminococcus fermentans, Eubacterium ventriosum, Bacteroides fragilis ss. fragilis, Bacteroides AR, Coprococcus catus, Aerostipes hadrus, Eubacterium cylindroides, Eubacterium ruminantium, Eubacterium CH-1, Staphylococcus epidermidis, Peptostreptococcus BL, Eubacterium limosum, Tissirella praeacuta, Bacteroides L, Fusobacterium mortiferum I, Fusobacterium naviforme, Clostridium innocuum, Clostridium ramosum, Propionibacterium acnes, Ruminococcus flavefaciens, Ruminococcus AT, Peptococcus AU-1, Bacteroides fragilis ss. ovatus, -ss. d, -ss. f; Bacteroides L-1, L-5; Fusobacterium nucleatum, Fusobacterium mortiferum, Escherichia coli, Gemella morbillorum, Finegoldia magnus, Peptococcus G, -AU-2; Streptococcus intermedius, Ruminococcus lactaris, Ruminococcus CO Gemmiger X, Coprococcus BH, -CC; Eubacterium tenue, Eubacterium ramulus, Bacteroides clostridiiformis ss. clostridliformis, Bacteroides coagulans, Prevotella oralis, Prevotella ruminicola, Odoribacter splanchnicus, Desuifomonas pigra, Lactobacillus G, Succinivibrio A, and a combination thereof.

In some aspects, pharmaceutical compositions provided herein comprise non-pathogenic Clostridia spores and viable non-pathogenic Collinsella without organisms from at least one of the groups consisting of Bacteroides, Fusobacteria, Propionibacteria, Lactobacilli, anaerobic cocci, Ruminococcus, E. coli, Gemmiger, Desulfomonas, Peptostreptococcus, and Bifidobacteria. In other aspects, pharmaceutical compositions comprise no viable non-pathogenic microorganisms selected from the group consisting of a Bacteroides fragilis ss. Vulgatus, Collinsella aerofaciens, Bacteroides fragilis ss. Thetaiotaomicron, Peptostreptococcus productus II, Parabacteroides distasonis Fusobacterium prausnitzii, Coprococcus eutactus, Collinsella aerofaciens III, Peptostreptococcus productus I, Ruminococcus bromii, Bifidobacterium adolescentis, Gemmiger formicilis, Bifdobacterium longum, Eubacterium siraeum, Ruminococcus torques, Eubacterium rectale, Eubacterium eligens, Bacteroides eggerthii, Clostridium leptum, Bacteroides fragilis ss. A, Eubacterium biforme, Bifidobacterium infantis, Eubacterium rectale III-F, Coprococcus comes, Pseudoflavonifractor capillosus, Ruminococcus albus, Dorea formicigenerans, Eubacterium hallii, Eubacterium ventriosum I, Fusobacterium russi, Ruminococcus obeum, Eubacterium rectale, Clostridium ramosum, Lactobacillus leichmannii, Ruminococcus callidus, Butyrivibrio crossotus, Acidaminococcus fermentans, Eubacterium ventriosum, Bacteroides fragilis ss. fragilis, Bacteroides AR, Coprococcus catus, Aerostipes hadrus, Eubacterium cylindroides, Eubacterium ruminantium, Eubacterium CH-1, Staphylococcus epidermidis, Peptostreptococcus BL, Eubacterium limosum, Tissirella praeacuta, Bacteroides L, Fusobacterium mortiferum I, Fusobacterium naviforme, Clostridium innocuum, Clostridium ramosum, Propionibacterium acnes, Ruminococcus flavefaciens, Ruminococcus AT, Peptococcus AU-1, Bacteroides fragilis ss. ovatus, -ss. d, -ss. f; Bacteroides L-1, L-5; Fusobacterium nucleatum, Fusobacterium mortiferum, Escherichia coli, Gemella morbillorum, Finegoldia magnus, Peptococcus G, -AU-2; Streptococcus intermedius, Ruminococcus lactaris, Ruminococcus CO Gemmiger X, Coprococcus BH, -CC; Eubacterium tenue, Eubacterium ramulus, Bacteroides clostridiiformis ss. clostridliformis, Bacteroides coagulans, Prevotella oralis, Prevotella ruminicola, Odoribacter splanchnicus, Desuifomonas pigra, Lactobacillus G, Succinivibrio A, and a combination thereof.

In one aspect, the transplantation product (e.g., a composition provided herein) is delivered by an infusion, e.g., through the rectum, stoma or down the upper gastrointestinal (GI) tract, or it can be used in a suppository pill, tablet or encapsulated form, e.g., with an enteric-coated graded release capsule or a tablet, e.g., with the addition of excipients. In one aspect, the transplantation product is administered as a suppository to give the highest concentration in the rectum.

In one aspect, the transplantation product (e.g., a composition provided herein, e.g., comprising an isolated or purified fecal flora or an entire (or substantially entire) microbiota) is stored at room temperature before or during delivery to an individual, e.g., in a fluid, e.g., a sterile fluid, such as saline, a buffer or a media such as a fluid-glucose-cellobiose agar (RGCA) media.

In one aspect, the compositions provided herein are used to ameliorate, stabilize, prevent and/or treat: various gastrointestinal conditions, e.g., C. difficile infection, C. perfringens welchii and other Clostridium infections, irritable bowel syndrome, constipation, pouchitis, Crohn's disease and microscopic colitis; neurological conditions such as autism, Parkinson's disease, myoclonus dystonia, autism, amyotrophic lateral sclerosis and multiple sclerosis, Grand mal seizures or petit mal seizures. In one aspect, the neurological conditions are treated by encapsulated or frozen material. In one aspect, for colitis patients, recurrent administration is required to suppress and reverse the inflammatory bowel disease and irritable bowel syndrome.

In one aspect, a crude collected stool is filtered and/or homogenized, and then its bacterial cells are separated (e.g., from the “crud” which contains the undigested food and fiber) by plasmapheresis, centrifugation, celltrifuge, column chromatography (e.g., affinity chromatography), immunoprecipitation (e.g., antibodies fixed to a solid surface, such as beads or a plate). Centrifugation, including use of a “celltrifuge” (e.g., a Baxter model MEDIFUGE 1215™) are processes that involve centrifugal force to separate mixtures. For “celltrifugation”, the densest components will then fly to the outside of the spinning plates while the rest of the components will migrate to the axis. The effect of the gravitational force will be increased by spinning the flattened product between rapidly moving glass plates. The centrifuge or celltrifuge can be set up such that the stool will be diluted adequately and set on a spinning cycle and collection of cells will occur only peripherally on the centrifuge.

In one aspect, wild type bacterial cells (including e.g., an entire (or substantially entire) microbiota) separated or purified e.g., by centrifugation, celltrifugation, plasmapheresis and the like. In one aspect, this material is stored at room temperature in a container, e.g., a bag, which can then be used to infuse through a colonoscope, naso-duodenal or nasogastric tube. In one aspect, it can be delivered to a facility (e.g., a hospital pharmacy) to be kept at room temperature, e.g., at between about 20° C. to 26° C. In one aspect, compositions provided herein are used either in a solution, gels, geltabs, pills, capsules or tablets, or suppositories, e.g., to be reconstituted later as an enema or infuse set through a colonoscope.

In one aspect, solutions, gels, geltabs, pills, capsules or tablets comprising compositions provided herein (e.g., isolated or purified fecal flora or an entire (or substantially entire) microbiota) can be taken long term, e.g., on a daily basis long term, e.g., for one, two, three or four weeks or months or more, to treat, stabilize, ameliorate or prevent a chronic and/or an immune condition such as e.g., autism, persistent infection, rheumatoid arthritis, systemic lupus erythematosus, autoimmune renal diseases, e.g., nephritis, severe obstruction, inflammatory bowel disease (IBD), irritable bowel syndrome (IBS), and other conditions set forth herein.

In one aspect, this application provides for the following embodiments:
Embodiment 1. A pharmaceutical composition comprising a lyophilized fecal microbe preparation comprising a lyophilization formulation comprising at least about 10% trehalose, wherein said lyophilization formulation is capable of providing a cell integrity maintenance ratio of at least about 20%.
Embodiment 2. The pharmaceutical composition of embodiment 1, wherein said lyophilization formulation comprises at least about 12.5%, at least about 13%, at least about 13.5%, at least about 14%, at least about 14.5%, at least about 15%, at least about 15.5%, at least about 16%, at least about 16.5%, at least about 17%, at least about 17.5%, at least about 18%, at least about 18.5%, at least about 19%, at least about 19.5%, at least about 20%, at least about 22.5%, at least about 25%, at least about 27.5%, at least about 30%, at least about 32.5%, at least about 35%, at least about 37.5%, at least about 40%, at least about 42.5%, at least about 45%, at least about 47.5%, at least about 50%, at least about 52.5%, at least about 55%, at least about 57.5%, or at least about 60% trehalose.
Embodiment 3. The pharmaceutical composition of embodiment 1, wherein said lyophilization formulation comprises between about 12.5% and about 60%, between about 13% and about 60%, between about 13.5% and about 60%, between about 14% and about 60%, between about 14.5% and about 60%, between about 15% and about 60%, between about 15.5% and about 60%, between about 16% and about 60%, between about 16.5% and about 60%, between about 17% and about 60%, between about 17.5% and about 60%, between about 18% and about 60%, between about 18.5% and about 60%, between about 19% and about 60%, between about 19.5% and about 60%, between about 20% and about 60%, between about 22.5% and about 60%, between about 25% and about 60%, between about 27.5% and about 60%, between about 30% and about 60%, between about 32.5% and about 60%, between about 35% and about 60%, or between about 37.5% and about 60% trehalose.
Embodiment 4. The pharmaceutical composition of embodiment 1, wherein said lyophilization formulation comprises between about 12.5% and about 40%, between about 13% and about 40%, between about 13.5% and about 40%, between about 14% and about 40%, between about 14.5% and about 40%, between about 15% and about 40%, between about 15.5% and about 40%, between about 16% and about 40%, between about 16.5% and about 40%, between about 17% and about 40%, between about 17.5% and about 40%, between about 18% and about 40%, between about 18.5% and about 40%, between about 19% and about 40%, between about 19.5% and about 40%, between about 20% and about 40%, between about 22.5% and about 40%, between about 25% and about 40%, between about 27.5% and about 40%, between about 30% and about 40%, between about 32.5% and about 40%, between about 35% and about 40%, or between about 37.5% and about 40% trehalose.
Embodiment 5. The pharmaceutical composition of embodiment 1, wherein said lyophilization formulation comprises between about 12.5% and about 30%, between about 13% and about 30%, between about 13.5% and about 30%, between about 14% and about 30%, between about 14.5% and about 30%, between about 15% and about 30%, between about 15.5% and about 30%, between about 16% and about 30%, between about 16.5% and about 30%, between about 17% and about 30%, between about 17.5% and about 30%, between about 18% and about 30%, between about 18.5% and about 30%, between about 19% and about 30%, between about 19.5% and about 30%, between about 20% and about 30%, between about 22.5% and about 30%, between about 25% and about 30%, or between about 27.5% and about 30% trehalose.
Embodiment 6. The pharmaceutical composition of embodiment 1, wherein said lyophilization formulation comprises between about 12.5% and about 25%, between about 13% and about 25%, between about 13.5% and about 25%, between about 14% and about 25%, between about 14.5% and about 25%, between about 15% and about 25%, between about 15.5% and about 25%, between about 16% and about 25%, between about 16.5% and about 25%, between about 17% and about 25%, between about 17.5% and about 25%, between about 18% and about 25%, between about 18.5% and about 25%, between about 19% and about 25%, between about 19.5% and about 25%, between about 20% and about 25%, or between about 22.5% and about 25% trehalose.
Embodiment 7. The pharmaceutical composition of embodiment 1, wherein said lyophilization formulation comprises between about 12.5% and about 20%, between about 13% and about 20%, between about 13.5% and about 20%, between about 14% and about 20%, between about 14.5% and about 20%, between about 15% and about 20%, between about 15.5% and about 20%, between about 16% and about 20%, between about 16.5% and about 20%, between about 17% and about 20%, between about 17.5% and about 20%, between about 18% and about 20%, between about 18.5% and about 20%, between about 19% and about 20%, or between about 19.5% and about 20% trehalose.
Embodiment 8. The pharmaceutical composition of embodiment 1, wherein said lyophilization formulation comprises between about 12.5% and about 17.5%, between about 13% and about 17.5%, between about 13.5% and about 17.5%, between about 14% and about 17.5%, between about 14.5% and about 17.5%, between about 15% and about 17.5%, between about 15.5% and about 17.5%, between about 16% and about 17.5%, between about 16.5% and about 17.5%, or between about 17% and about 17.5% trehalose.
Embodiment 9. The pharmaceutical composition of embodiment 1, wherein said lyophilization formulation comprises between about 12.5% and about 17.5%, between about 13% and about 17%, between about 13.5% and about 16.5%, between about 14% and about 16%, or between about 14.5% and about 15.5% trehalose.
Embodiment 10. The pharmaceutical composition of embodiment 1, wherein said lyophilization formulation comprises about 15% trehalose.
Embodiment 11. The pharmaceutical composition of embodiment 1, wherein said lyophilization formulation comprises 15% trehalose.
Embodiment 12. The pharmaceutical composition of any one of embodiments 1 to 11, wherein said lyophilization formulation is capable of providing a cell integrity maintenance ratio of at least about 22.5%, at least about 25%, at least about 27.5%, at least about 30%, at least about 32.5%, at least about 35%, at least about 37.5%, at least about 40%, at least about 42.5%, at least about 45%, at least about 47.5%, at least about 50%, at least about 52.5%, at least about 55%, at least about 57.5%, at least about 60%, at least about 62.5%, at least about 65%, at least about 67.5%, at least about 70%, at least about 72.5%, at least about 75%, at least about 77.5%, at least about 80%, at least about 82.5%, at least about 85%, at least about 87.5%, at least about 90%, at least about 92.5%, or at least about 95%.
Embodiment 13. The pharmaceutical composition of any one of embodiments 1 to 11, wherein said lyophilization formulation is capable of providing a cell integrity maintenance ratio of between about 22.5% and about 90%, of between about 25% and about 90%, of between about 27.5% and about 90%, of between about 30% and about 90%, between about 32.5% and about 90%, between about 35% and about 90%, between about 37.5% and about 90%, between about 40% and about 90%, between about 42.5% and about 90%, between about 45% and about 90%, between about 47.5% and about 90%, between about 50% and about 90%, between about 52.5% and about 90%, between about 55% and about 90%, between about 57.5% and about 90%, between about 60% and about 90%, between about 62.5% and about 90%, between about 65% and about 90%, between about 67.5% and about 90%, between about 70% and about 90%, between about 72.5% and about 90%, between about 75% and about 90%, between about 77.5% and about 90%, between about 80% and about 90%, between about 82.5% and about 90%, between about 85% and about 90%, or between about 87.5% and about 90%.
Embodiment 14. The pharmaceutical composition of any one of embodiments 1 to 11, wherein said lyophilization formulation is capable of providing a cell integrity maintenance ratio of between about 22.5% and about 80%, of between about 25% and about 80%, of between about 27.5% and about 80%, of between about 30% and about 80%, between about 32.5% and about 80%, between about 35% and about 80%, between about 37.5% and about 80%, between about 40% and about 80%, between about 42.5% and about 80%, between about 45% and about 80%, between about 47.5% and about 80%, between about 50% and about 80%, between about 52.5% and about 80%, between about 55% and about 80%, between about 57.5% and about 80%, between about 60% and about 80%, between about 62.5% and about 80%, between about 65% and about 80%, between about 67.5% and about 80%, between about 70% and about 80%, between about 72.5% and about 80%, between about 75% and about 80%, or between about 77.5% and about 80%.
Embodiment 15. The pharmaceutical composition of any one of embodiments 1 to 11, wherein said lyophilization formulation is capable of providing a cell integrity maintenance ratio of between about 22.5% and about 70%, of between about 25% and about 70%, of between about 27.5% and about 70%, of between about 30% and about 70%, between about 32.5% and about 70%, between about 35% and about 70%, between about 37.5% and about 70%, between about 40% and about 70%, between about 42.5% and about 70%, between about 45% and about 70%, between about 47.5% and about 70%, between about 50% and about 70%, between about 52.5% and about 70%, between about 55% and about 70%, between about 57.5% and about 70%, between about 60% and about 70%, between about 62.5% and about 70%, between about 65% and about 70%, or between about 67.5% and about 70%.
Embodiment 16. The pharmaceutical composition of any one of embodiments 1 to 11, wherein said lyophilization formulation is capable of providing a cell integrity maintenance ratio of between about 22.5% and about 60%, of between about 25% and about 60%, of between about 27.5% and about 60%, of between about 30% and about 60%, between about 32.5% and about 60%, between about 35% and about 60%, between about 37.5% and about 60%, between about 40% and about 60%, between about 42.5% and about 60%, between about 45% and about 60%, between about 47.5% and about 60%, between about 50% and about 60%, between about 52.5% and about 60%, between about 55% and about 60%, or between about 57.5% and about 60%.
Embodiment 17. The pharmaceutical composition of any one of embodiments 1 to 11, wherein said lyophilization formulation is capable of providing a cell integrity maintenance ratio of between about 22.5% and about 50%, of between about 25% and about 50%, of between about 27.5% and about 50%, of between about 30% and about 50%, between about 32.5% and about 50%, between about 35% and about 50%, between about 37.5% and about 50%, between about 40% and about 50%, between about 42.5% and about 50%, between about 45% and about 50%, or between about 47.5% and about 50%.
Embodiment 18. The pharmaceutical composition of any one of embodiments 1 to 11, wherein said lyophilization formulation is capable of providing a cell integrity maintenance ratio between about 22.5% and about 25%, between about 27.5% and about 30%, between about 30% and 32.5%, between about 32.5% and about 80%, between about 35% and about 77.5%, between about 37.5% and about 75%, between about 40% and about 67.5%, between about 42.5% and about 65%, between about 45% and about 62.5%, between about 47.5% and about 60%, between about 50% and about 57.5%, or between about 52.5% and about 55%.
Embodiment 19. The pharmaceutical composition of any one of embodiments 1 to 11, wherein said lyophilization formulation is capable of providing a cell integrity maintenance ratio of between about 32.5% and about 90%, between about 35% and about 87.5%, between about 37.5% and about 85%, between about 40% and about 82.5%, between about 42.5% and about 77.5%, between about 45% and about 75%, between about 47.5% and about 72.5%, between about 50% and about 70%, between about 52.5% and about 67.5%, between about 55% and about 65%, or between about 57.5% and about 62.5%.
Embodiment 20. The pharmaceutical composition of any one of embodiments 1 to 11, wherein said lyophilization formulation is capable of a post-lyophilization cell membrane integrity percentage of at least about 22.5%, at least about 25%, at least about 27.5%, at least about 30%, at least about 32.5%, at least about 35%, at least about 37.5%, at least about 40%, at least about 42.5%, at least about 45%, at least about 47.5%, at least about 50%, at least about 52.5%, at least about 55%, at least about 57.5%, at least about 60%, at least about 62.5%, at least about 65%, at least about 67.5%, at least about 70%, at least about 72.5%, at least about 75%, at least about 77.5%, at least about 80%.
Embodiment 21. The pharmaceutical composition of any one of embodiments 1 to 11, wherein said lyophilization formulation is capable of a post-lyophilization cell membrane integrity percentage of between about 20% and about 80%, between about 22.5% and about 80%, between about 25% and about 80%, between about 27.5% and about 80%, between about 30% and about 80%, between about 32.5% and about 80%, between about 35% and about 80%, between about 37.5% and about 80%, between about 40% and about 80%, between about 42.5% and about 80%, between about 45% and about 80%, between about 47.5% and about 80%, between about 50% and about 80%, between about 52.5% and about 80%, between about 55% and about 80%, between about 57.5% and about 80%, between about 60% and about 80%, between about 62.5% and about 80%, between about 65% and about 80%, between about 67.5% and about 80%, between about 70% and about 80%, between about 72.5% and about 80%, between about 75% and about 80%, or between about 77.5% and about 80%.
Embodiment 22. The pharmaceutical composition of any one of embodiments 1 to 11, wherein said lyophilization formulation is capable of a post-lyophilization cell membrane integrity percentage of between about 20% and about 70%, between about 22.5% and about 70%, between about 25% and about 70%, between about 27.5% and about 70%, between about 30% and about 70%, between about 32.5% and about 70%, between about 35% and about 70%, between about 37.5% and about 70%, between about 40% and about 70%, between about 42.5% and about 70%, between about 45% and about 70%, between about 47.5% and about 70%, between about 50% and about 70%, between about 52.5% and about 70%, between about 55% and about 70%, between about 57.5% and about 70%, between about 60% and about 70%, between about 62.5% and about 70%, between about 65% and about 70%, or between about 67.5% and about 70%.
Embodiment 23. The pharmaceutical composition of any one of embodiments 1 to 11, wherein said lyophilization formulation is capable of a post-lyophilization cell membrane integrity percentage of between about 20% and about 60%, between about 22.5% and about 60%, between about 25% and about 60%, between about 27.5% and about 60%, between about 30% and about 60%, between about 32.5% and about 60%, between about 35% and about 60%, between about 37.5% and about 60%, between about 40% and about 60%, between about 42.5% and about 60%, between about 45% and about 60%, between about 47.5% and about 60%, between about 50% and about 60%, between about 52.5% and about 60%, between about 55% and about 60%, or between about 57.5% and about 60%.
Embodiment 24. The pharmaceutical composition of any one of embodiments 1 to 11, wherein said lyophilization formulation is capable of a post-lyophilization cell membrane integrity percentage of between about 20% and about 50%, between about 22.5% and about 50%, between about 25% and about 50%, between about 27.5% and about 50%, between about 30% and about 50%, between about 32.5% and about 50%, between about 35% and about 50%, between about 37.5% and about 50%, between about 40% and about 50%, between about 42.5% and about 50%, between about 45% and about 50%, or between about 47.5% and about 50%.
Embodiment 25. The pharmaceutical composition of any one of embodiments 1 to 11, wherein said lyophilization formulation is capable of a post-lyophilization cell membrane integrity percentage of between about 20% and about 40%, between about 22.5% and about 40%, between about 25% and about 40%, between about 27.5% and about 40%, between about 30% and about 40%, between about 32.5% and about 40%, between about 35% and about 40%, or between about 37.5% and about 40%.
Embodiment 26. The pharmaceutical composition of any one of embodiments 1 to 11, wherein said lyophilization formulation is capable of a post-lyophilization cell membrane integrity percentage of between about 32.5% and about 80%, between about 35% and about 77.5%, between about 37.5% and about 75%, between about 40% and about 67.5%, between about 42.5% and about 65%, between about 45% and about 62.5%, between about 47.5% and about 60%, between about 50% and about 57.5%, or between about 52.5% and about 55%.
Embodiment 27. The pharmaceutical composition of any one of embodiments 1 to 11, wherein said lyophilization formulation is capable of a post-lyophilization cell membrane integrity percentage of between about 32.5% and about 90%, between about 35% and about 87.5%, between about 37.5% and about 85%, between about 40% and about 82.5%, between about 42.5% and about 77.5%, between about 45% and about 75%, between about 47.5% and about 72.5%, between about 50% and about 70%, between about 52.5% and about 67.5%, between about 55% and about 65%, or between about 57.5% and about 62.5%.
Embodiment 28. The pharmaceutical composition of any one of embodiments 1 to 11, wherein said lyophilization formulation is capable of providing a post-lyophilization cell membrane integrity percentage of between about 25% and about 55%, between about 27.5% and about 52.5%, between about 30% and about 50%, between about 32.5% and about 47.5%, between about 35% and about 45%, or between about 37.5% and about 42.5%.
Embodiment 29. The pharmaceutical composition of any one of embodiments 1 to 28, wherein said lyophilization formulation further comprises a reducing agent
Embodiment 30. The pharmaceutical composition of embodiment 29, wherein said reducing agent is cysteine selected from the group consisting of D-cysteine and L-cysteine, or sodium abscorbate.
Embodiment 31. The pharmaceutical composition of embodiment 30, wherein said cysteine is at a concentration of at least about 0.025%.
Embodiment 32. The pharmaceutical composition of embodiment 30, wherein said cysteine is at a concentration of about 0.025%.
Embodiment 33. The pharmaceutical composition of embodiment 30, wherein said cysteine is at a concentration of 0.025%.
Embodiment 34. The pharmaceutical composition of embodiment 30, wherein said cysteine is at a concentration of at least about 0.001%, at least about 0.005%, at least about 0.01%, at least about 0.015%, at least about 0.02%, at least about 0.025%, at least about 0.03%, at least about 0.035%, at least about 0.04%, at least about 0.045%, at least about 0.05%, at least about 0.055%, at least about 0.06%, at least about 0.065%, at least about 0.07%, at least about 0.075%, at least about 0.08%, at least about 0.085%, at least about 0.09%, at least about 0.095%, at least about 0.1%, at least about 0.12%, at least about 0.14%, at least about 0.16%, at least about 0.18%, at least about 0.2%, at least about 0.25%, at least about 0.3%, at least about 0.4%, at least about 0.5%, at least about 0.6%, at least about 0.7%, at least about 0.8%, at least about 0.9%, at least about 1%, at least about 2%, at least about 4%, at least about 6%, at least about 8%, at least about 10%, at least about 12%, at least about 14%, at least about 16%, at least about 18%, at least about 20%, at least about 22%, at least about 24%, or at least about 26%.
Embodiment 35. The pharmaceutical composition of embodiment 30, wherein said cysteine is at a concentration of between about 0.005% and about 0.1%, between about 0.01% and about 0.1%, between about 0.015% and about 0.1%, between about 0.02% and about 0.1%, between about 0.025% and about 0.1%, between about 0.03% and about 0.1%, between about 0.035% and about 0.1%, between about 0.04% and about 0.1/%, between about 0.045% and about 0.1%, between about 0.05% and about 0.1%, between about 0.055% and about 0.1%, between about 0.06% and about 0.1%, between about 0.065% and about 0.1%, between about 0.07% and about 0.1%, between about 0.075% and about 0.1%, between about 0.08% and about 0.1%, between about 0.085% and about 0.1%, between about 0.09% and about 0.1%, between about 0.095% and about 0.1%.
Embodiment 36. The pharmaceutical composition of embodiment 30, wherein said cysteine is at a concentration of between about 0.005% and about 0.08%, between about 0.01% and about 0.08%, between about 0.015% and about 0.08%, between about 0.02% and about 0.08%, between about 0.025% and about 0.08%, between about 0.03% and about 0.08%, between about 0.035% and about 0.08%, between about 0.04% and about 0.08%, between about 0.045% and about 0.08%, between about 0.05% and about 0.08%, between about 0.055% and about 0.08%, between about 0.06% and about 0.08%, between about 0.065% and about 0.08%, between about 0.07% and about 0.08%, or between about 0.075% and about 0.08%.
Embodiment 37. The pharmaceutical composition of embodiment 30, wherein said cysteine is at a concentration of between about 0.005% and about 0.06%, between about 0.01% and about 0.06%, between about 0.015% and about 0.06%, between about 0.02% and about 0.06%, between about 0.025% and about 0.06%, between about 0.03% and about 0.06%, between about 0.035% and about 0.06%, between about 0.04% and about 0.06%, between about 0.045% and about 0.06%, between about 0.05% and about 0.06%, or between about 0.055% and about 0.06%.
Embodiment 38. The pharmaceutical composition of embodiment 30, wherein said cysteine is at a concentration of between about 0.005% and about 0.04/0, between about 0.01% and about 0.04%, between about 0.015% and about 0.04%, between about 0.02% and about 0.04%, between about 0.025% and about 0.04%, between about 0.03% and about 0.04%, or between about 0.035% and about 0.04%.
Embodiment 39. The pharmaceutical composition of embodiment 30, wherein said cysteine is at a concentration of between about 0.005% and about 0.06%, between about 0.01% and about 0.05%, between about 0.015% and about 0.04%, or between about 0.02% and about 0.03%.
Embodiment 40. The pharmaceutical composition of any one of the preceding embodiments, wherein said fecal microbe preparation comprises a non-selected and substantially complete fecal microbiota preparation from a single donor.
Embodiment 41. The pharmaceutical composition of embodiment 40, wherein the weight ratio between fecal-derived non-living material and fecal-derived biological material in said fecal microbiota preparation is no greater than 20%.
Embodiment 42. The pharmaceutical composition of embodiment 40, wherein the preparation of said fecal microbe preparation involves a treatment selected from the group consisting of ethanol treatment, detergent treatment, heat treatment, irradiation, homogenization, sonication, and combination thereof.
Embodiment 43. The pharmaceutical composition of embodiment 40, wherein the preparation of said fecal microbe preparation involves a separation step selected from the group consisting of filtering, sieving, density gradients, filtration, chromatography, sedimentation, centrifugation, and a combination thereof.
Embodiment 44. The pharmaceutical composition of embodiment 40, wherein after at least 12 weeks of storage at ambient temperature or lower said fecal microbiota preparation is capable of maintaining at least 60% cell viability relative to the initial cell viability immediately prior to storage.
Embodiment 45. The pharmaceutical composition of embodiment 44, wherein after at least 12 weeks of storage at ambient temperature or lower said fecal microbiota preparation is capable of maintaining about 60% to about 80% cell viability relative to the initial cell viability at the start of said storage.
Embodiment 46. The pharmaceutical composition of embodiment 44, wherein the cell viability is measured by assessing cell membrane permeability via a combination of membrane permeant and impermeant DNA dyes stains or non-DNA binding dyes.
Embodiment 47. The pharmaceutical composition of embodiment 40, wherein said lyophilized formulation further comprises one or more cryoprotectants selected from the group consisting of dimethyl sulfoxide (DMSO), glycerol, polyethylene glycol (PEG), alanine, glycine, proline, mannitol, sucrose, glucose, lactose, ribose, hydroxypropyl-β-cyclodextrin (HPβCD), and any combination thereof.
Embodiment 48. The pharmaceutical composition of embodiment 40, wherein said pharmaceutical composition is for oral administration.
Embodiment 49. The pharmaceutical composition of embodiment 40, wherein said pharmaceutical composition is formulated as a geltab, pill, microcapsule, capsule, or tablet.
Embodiment 50. The pharmaceutical composition of embodiment 40, wherein every 200 mg of said pharmaceutical composition comprises a pharmacologically active dose of microbes or spores selected from the group consisting of 10³ to 10¹⁵, 10⁴ to 10¹⁵, 10⁵ to 10¹⁵, 10⁶ to 10¹⁵, 10⁷ to 10¹⁵, 10⁸ to 10¹⁵, 10³ to 10¹⁴, 10⁴ to 10¹⁴, 10⁵ to 10¹⁴, 10⁶ to 10¹⁴, 10⁷ to 10¹⁴, 10⁸ to 10¹⁴, 10⁴ to 10¹³, 10⁵ to 10¹², 10⁶ to 10¹¹, 10⁷ to 10¹⁰, 10⁸ to 10⁹, 10³ to 10¹³, 10³ to 10¹², 10³ to 10¹¹, 10³ to 10¹⁰, 10³ to 10⁹, 10³ to 10⁸, 10³ to 10⁷, 10³ to 10⁶, 10³ to 10⁵, and 10³ to 10⁴ cfu or total cell count.
Embodiment 51. The pharmaceutical composition of embodiment 40, wherein said fecal microbe preparation has at least about 10%, 20%, 30%, 40%, 50%, 60%, 70%, 80%, 85%, 90%, 95%, 99%, or 99.5% microbes in a spore form.
Embodiment 52. The pharmaceutical composition of embodiment 40, wherein said pharmaceutical composition is effective for treating one or more disorders selected from the group consisting of recurrent or primary C. diff infection, autism spectrum disorder (ASD), ulcerative colitis, Crohn's disease, and irritable bowel syndrome.
Embodiment 53. A lyophilized fecal microbe composition comprising a lyophilization formulation comprising trehalose and cysteine, wherein said lyophilization formulation is capable of maintaining at least about 30% cell viability immediately post lyophilization relative to a pre-lyophilization cell viability.
Embodiment 54. The lyophilized fecal microbe composition of embodiment 53, wherein said lyophilization formulation comprises at least 15% trehalose and at least 0.025% L-cysteine.
Embodiment 55. A lyophilized fecal microbiota pharmaceutical composition comprising a lyophilization formulation comprising at least about 12.5% trehalose, wherein said lyophilization formulation is capable of providing a cell integrity maintenance ratio of at least 35%.
Embodiment 56. A method of preparing a lyophilized fecal microbe preparation, said method comprising: a. obtaining a liquid fecal microbe preparation in a lyophilization formulation comprising between 10% and 20% trehalose; and b. freeze-drying said liquid fecal microbe preparation, wherein said lyophilization formulation is capable of providing a cell integrity maintenance ratio of at least about 30%.
Embodiment 57. The method of embodiment 56, wherein said lyophilization formulation comprises between 12.5% and 17.5% trehalose.
Embodiment 58. The method of embodiment 56, wherein said lyophilization formulation comprises at least about 15% trehalose.
Embodiment 59. The method of embodiment 56, wherein said lyophilization formulation comprises about 15% trehalose.
Embodiment 60. The method of embodiment 56, wherein said lyophilization formulation further comprises L-cysteine.
Embodiment 61. The method of embodiment 60, wherein said lyophilization formulation comprises at least about 0.02% L-cysteine.
Embodiment 62. The method of embodiment 60, wherein said lyophilization formulation comprises about 0.025% L-cysteine.
Embodiment 63. A frozen fecal microbe composition comprising trehalose, wherein said frozen fecal microbe composition is capable of being thawed at least once without loss of microbial cell membrane integrity of more than 50%.
Embodiment 64. The frozen fecal microbe composition of embodiment 63, wherein said being thawed at least once is thawed at least 2, 3, 4, 5, or 6 times.
Embodiment 65. The frozen fecal microbe composition of embodiment 63, wherein said frozen fecal microbe composition is capable of being thawed at least once without loss of microbial cell membrane integrity of more than 40%, more than 35%, more than 30%, more than 25%, more than 20%, more than 15%, more than 10%, or more than 5%.
Embodiment 66. The frozen fecal microbe composition of embodiment 63, wherein said composition comprises at least about 12.5%, at least about 13%, at least about 13.5%, at least about 14%, at least about 14.5%, at least about 15%, at least about 15.5%, at least about 16%, at least about 16.5%, at least about 17%, at least about 17.5%, at least about 18%, at least about 18.5%, at least about 19%, at least about 19.5%, at least about 20%, at least about 22.5%, at least about 25%, at least about 27.5%, at least about 30%, at least about 32.5%, at least about 35%, at least about 37.5%, at least about 40%, at least about 42.5%, at least about 45%, at least about 47.5%, at least about 50%, at least about 52.5%, at least about 55%, at least about 57.5%, or at least about 60% trehalose.
Embodiment 67. The frozen fecal microbe composition of embodiment 63, wherein said composition comprises between about 12.5% and about 60%, between about 13% and about 60%, between about 13.5% and about 60%, between about 14% and about 60%, between about 14.5% and about 60%, between about 15% and about 60%, between about 15.5% and about 60%, between about 16% and about 60%, between about 16.5% and about 60%, between about 17% and about 60%, between about 17.5% and about 60%, between about 18% and about 60%, between about 18.5% and about 60%, between about 19% and about 60%, between about 19.5% and about 60%, between about 20% and about 60%, between about 22.5% and about 60%, between about 25% and about 60%, between about 27.5% and about 60%, between about 30% and about 60%, between about 32.5% and about 60%, between about 35% and about 60%, or between about 37.5% and about 60% trehalose.
Embodiment 68. The frozen fecal microbe composition of embodiment 63, wherein said composition comprises between about 12.5% and about 40%, between about 13% and about 40%, between about 13.5% and about 40%, between about 14% and about 40%, between about 14.5% and about 40%, between about 15% and about 40%, between about 15.5% and about 40%, between about 16% and about 40%, between about 16.5% and about 40%, between about 17% and about 40%, between about 17.5% and about 40%, between about 18% and about 40%, between about 18.5% and about 40%, between about 19% and about 40%, between about 19.5% and about 40%, between about 20% and about 40%, between about 22.5% and about 40%, between about 25% and about 40%, between about 27.5% and about 40%, between about 30% and about 40%, between about 32.5% and about 40%, between about 35% and about 40%, or between about 37.5% and about 40% trehalose.
Embodiment 69. The frozen fecal microbe composition of embodiment 63, wherein said composition comprises between about 12.5% and about 30%, between about 13% and about 30%, between about 13.5% and about 30%, between about 14% and about 30%, between about 14.5% and about 30%, between about 15% and about 30%, between about 15.5% and about 30%, between about 16% and about 30%, between about 16.5% and about 30%, between about 17% and about 30%, between about 17.5% and about 30%, between about 18% and about 30%, between about 18.5% and about 30%, between about 19% and about 30%, between about 19.5% and about 30%, between about 20% and about 30%, between about 22.5% and about 30%, between about 25% and about 30%, or between about 27.5% and about 30% trehalose.
Embodiment 70. The frozen fecal microbe composition of embodiment 63, wherein said composition comprises between about 12.5% and about 25%, between about 13% and about 25%, between about 13.5% and about 25%, between about 14% and about 25%, between about 14.5% and about 25%, between about 15% and about 25%, between about 15.5% and about 25%, between about 16% and about 25%, between about 16.5% and about 25%, between about 17% and about 25%, between about 17.5% and about 25%, between about 18% and about 25%, between about 18.5% and about 25%, between about 19% and about 25%, between about 19.5% and about 25%, between about 20% and about 25%, or between about 22.5% and about 25% trehalose.
Embodiment 71. The frozen fecal microbe composition of embodiment 63, wherein said composition comprises between about 12.5% and about 20%, between about 13% and about 20%, between about 13.5% and about 20%, between about 14% and about 20%, between about 14.5% and about 20%, between about 15% and about 20%, between about 15.5% and about 20%, between about 16% and about 20%, between about 16.5% and about 20%, between about 17% and about 20%, between about 17.5% and about 20%, between about 18% and about 20%, between about 18.5% and about 20%, between about 19% and about 20%, or between about 19.5% and about 20% trehalose.
Embodiment 72. The frozen fecal microbe composition of embodiment 63, wherein said composition comprises between about 12.5% and about 17.5%, between about 13% and about 17.5%, between about 13.5% and about 17.5%, between about 14% and about 17.5%, between about 14.5% and about 17.5%, between about 15% and about 17.5%, between about 15.5% and about 17.5%, between about 16% and about 17.5%, between about 16.5% and about 17.5%, or between about 17% and about 17.5% trehalose.
Embodiment 73. The frozen fecal microbe composition of embodiment 63, wherein said composition comprises between about 12.5% and about 17.5%, between about 13% and about 17%, between about 13.5% and about 16.5%, between about 14% and about 16%, or between about 14.5% and about 15.5% trehalose.
Embodiment 74. The frozen fecal microbe composition of embodiment 63, wherein said composition comprises about 15% trehalose.
Embodiment 75. The frozen fecal microbe composition of embodiment 63, wherein said composition comprises 15% trehalose.
Embodiment 76. The frozen fecal microbe composition of any one of embodiments 63 to 75, wherein said composition further comprises cysteine selected from the group consisting of D-cysteine and L-cysteine.
Embodiment 77. The frozen fecal microbe composition of embodiment 76, wherein said cysteine is at a concentration of at least about 0.025%.
Embodiment 78. The frozen fecal microbe composition of embodiment 76, wherein said cysteine is at a concentration of about 0.025%.
Embodiment 79. The frozen fecal microbe composition of embodiment 76, wherein said cysteine is at a concentration of 0.025%.
Embodiment 80. The frozen fecal microbe composition of embodiment 76, wherein said cysteine is at a concentration of at least about 0.005%, at least about 0.01%, at least about 0.015%, at least about 0.02%, at least about 0.025%, at least about 0.03%, at least about 0.035%, at least about 0.04%, at least about 0.045%, at least about 0.05%, at least about 0.055%, at least about 0.06%, at least about 0.065%, at least about 0.07%, at least about 0.075%, at least about 0.08%, at least about 0.085%, at least about 0.09%, at least about 0.095%, at least about 0.1%, at least about 0.12%, at least about 0.14%, at least about 0.16%, at least about 0.18%, at least about 0.2%, at least about 0.25%, at least about 0.3%, at least about 0.4%, at least about 0.5%, at least about 0.6%, at least about 0.7%, at least about 0.8%, at least about 0.9%, at least about 1%, at least about 2%, at least about 4%, at least about 6%, at least about 8%, at least about 10%, at least about 12%, at least about 14%, at least about 16%, at least about 18%, at least about 20%, at least about 22%, at least about 24%, or at least about 26%.
Embodiment 81. The frozen fecal microbe composition of embodiment 76, wherein said cysteine is at a concentration of between about 0.005% and about 0.1%, between about 0.01% and about 0.1%, between about 0.015% and about 0.1%, between about 0.02% and about 0.1%, between about 0.025% and about 0.1%, between about 0.03% and about 0.1%, between about 0.035% and about 0.1%, between about 0.04% and about 0.1/%, between about 0.045% and about 0.1%, between about 0.05% and about 0.1%, between about 0.055% and about 0.1%, between about 0.06% and about 0.1%, between about 0.065% and about 0.1%, between about 0.07% and about 0.1%, between about 0.075% and about 0.1%, between about 0.08% and about 0.1%, between about 0.085% and about 0.1%, between about 0.09% and about 0.1%, between about 0.095% and about 0.1%.
Embodiment 82. The frozen fecal microbe composition of embodiment 76, wherein said cysteine is at a concentration of between about 0.005% and about 0.08%, between about 0.01% and about 0.08%, between about 0.015% and about 0.08%, between about 0.02% and about 0.08%, between about 0.025% and about 0.08%, between about 0.03% and about 0.08%, between about 0.035% and about 0.08%, between about 0.04% and about 0.08%, between about 0.045% and about 0.08%, between about 0.05% and about 0.08%, between about 0.055% and about 0.08%, between about 0.06% and about 0.08%, between about 0.065% and about 0.08%, between about 0.07% and about 0.08%, or between about 0.075% and about 0.08%.
Embodiment 83. The frozen fecal microbe composition of embodiment 76, wherein said cysteine is at a concentration of between about 0.005% and about 0.06%, between about 0.01% and about 0.06%, between about 0.015% and about 0.06%, between about 0.02% and about 0.06%, between about 0.025% and about 0.06%, between about 0.03% and about 0.06%, between about 0.035% and about 0.06%, between about 0.04% and about 0.06%, between about 0.045% and about 0.06%, between about 0.05% and about 0.06%, or between about 0.055% and about 0.06%.
Embodiment 84. The frozen fecal microbe composition of embodiment 76, wherein said cysteine is at a concentration of between about 0.005% and about 0.04%, between about 0.01% and about 0.04%, between about 0.015% and about 0.04%, between about 0.02% and about 0.04%, between about 0.025% and about 0.04%, between about 0.03% and about 0.04%, or between about 0.035% and about 0.04%.
Embodiment 85. The frozen fecal microbe composition of embodiment 76, wherein said cysteine is at a concentration of between about 0.005% and about 0.06%, between about 0.01% and about 0.05%, between about 0.015% and about 0.04%, or between about 0.02% and about 0.03%.
Embodiment 86. The frozen fecal microbe composition of any one of embodiments 63 to 85, wherein said fecal microbe composition comprises a non-selected and substantially complete fecal microbiota preparation from a single donor.
Embodiment 87. The frozen fecal microbe composition of embodiment 86, wherein the weight ratio between fecal-derived non-living material and fecal-derived biological material in said fecal microbiota preparation is no greater than 20%.
Embodiment 88. The frozen fecal microbe composition of embodiment 86, wherein the preparation of said fecal microbe preparation involves a treatment selected from the group consisting of ethanol treatment, detergent treatment, heat treatment, irradiation, and sonication, or a combination thereof.
Embodiment 89. The frozen fecal microbe composition of embodiment 86, wherein the preparation of said fecal microbe preparation involves a separation step selected from the group consisting of filtering, sieving, density gradients, filtration, chromatography, and a combination thereof.
Embodiment 90. The frozen fecal microbe composition of embodiment 86, wherein after at least 12 weeks of storage at ambient temperature or lower said fecal microbiota preparation is capable of maintaining at least 60% cell viability relative to the initial cell viability immediately prior to storage.
Embodiment 91. The frozen fecal microbe composition of embodiment 90, wherein after at least 12 weeks of storage at ambient temperature or lower said fecal microbiota preparation is capable of maintaining about 60% to about 80% cell viability relative to the initial cell viability at the start of said storage.
Embodiment 92. The frozen fecal microbe composition of embodiment 90, wherein the cell viability is measured by assessing cell membrane permeability via a combination of membrane permeant and impermeant DNA dyes stains.
Embodiment 93. The frozen fecal microbe composition of embodiment 63, wherein said composition further comprises one or more cryoprotectants selected from the group consisting of dimethyl sulfoxide (DMSO), glycerol, polyethylene glycol (PEG), alanine, glycine, proline, mannitol, sucrose, glucose, lactose, ribose, hydroxypropyl-β-cyclodextrin (HPβCD), and any combination thereof.
Embodiment 94. The frozen fecal microbe composition of embodiment 63, wherein said composition is for oral administration.
Embodiment 95. The frozen fecal microbe composition of embodiment 63, wherein said composition is formulated as a geltab, pill, microcapsule, capsule, or tablet Embodiment 96. The frozen fecal microbe composition of embodiment 63, wherein every 200 mg of said composition comprises a pharmacologically active dose of microbes or spores selected from the group consisting of 10³ to 10¹⁴, 10⁴ to 10¹⁴, 10⁵ to 10¹⁴, 10⁶ to 10¹⁴, 10⁷ to 10¹⁴, 10⁸ to 10¹⁴, 10⁴ to 10¹³, 10⁵ to 10¹², 10⁶ to 10¹¹, 10⁷ to 10¹⁰, 10⁸ to 10⁹, 10³ to 10¹³, 10³ to 10¹², 10³ to 10¹¹, 10³ to 10¹⁰, 10³ to 10⁹, 10³ to 10⁸, 10³ to 10⁷, 10³ to 10⁶, 10³ to 10⁵, and 10³ to 10⁴ cfu or total cell count.
Embodiment 97. The frozen fecal microbe composition of embodiment 63, wherein said fecal microbe composition has at least about 20%, 30%, 40%, 50%, 60%, 70%, 80%, 85%, 90%, 95%, 99%, or 99.5% microbes in a spore form.
Embodiment 98. The frozen fecal microbe composition of embodiment 63, wherein said composition is effective for treating one or more disorders selected from the group consisting of recurrent or primary C. diff. infection, autism spectrum disorder (ASD), ulcerative colitis, Crohn's disease, and irritable bowel syndrome.
Embodiment 99. A method for treating a disorder or condition in a subject in need thereof, comprising administering to said subject a therapeutically effective amount of a pharmaceutical composition of any one of embodiments 1 to 52 or a frozen fecal microbe composition of any one of embodiments 63 to 98, wherein said disorder or condition is selected from the group consisting of Acne, AIDS Enteropathy, AIDS-related Gastroenteritis, Alopecia Totalis, Alzheimers Disease, Amyloidosis, Amyotrophic Lateral Sclerosis, Ankylosing Spondylitis, Anorexia, Antibiotic Associated Colitis, Asbergers Syndrome, Attention Deficit Disorder (ADD), Attention Deficit Hyperactivity Disorder (ADHD), Autism Spectrum Disorder (ASD), Behcet's Syndrome, Chronic Clostridium difficile Infection (CDI), Chronic constipation, Chronic Depression, Chronic Fatigue Syndrome (CFS), Chronic Idiopathic Pseudo Obstructive Syndrome, Chronic Inflammation Demyelinating Polyneuropathy, Chronic Nausea, Chronic Urticaria, Coeliac Disease, Collagenous Colitis, Colonic Polyps, Constipation Predominant FBD, Crohn's Disease, Cryptogenic Cirrhosis, Cyclic Vomiting, Dermatitis Herpetiformis, Diabetes, Familial Mediterranean Fever, Fatty Liver, Functional Bowel Disease (FBD), Gastro-oesophageal Reflux, Gillian-Barre Syndrome, Glomerulonephritis, Haemolytic Uraemic Syndrome, Halitosis, IBS constipation-predominant, IBS diarrhea/constipation alternating, IBS diarrhea-predominant, IBS pain-predominant, Idiopathic Thrombocytopenic Purpura (ITP), Idiopathic/Simple Constipation, Indeterminate Colitis, Inflammatory Bowel Disease (IBD), Irritable bowel syndrome (IBS), Juvenile Diabetes Mellitus, Lyme Disease, Manic Depressive Illness, Metabolic Syndrome, Microscopic Colitis, Migraine, Mixed Cryoglobulinaemia, Mucous Colitis, Multiple Sclerosis, Myasthenia Gravis, NASH (Nonalcoholic Steatohepatitis), Non-Rheumatoid Arthritis, Non-Rheumatoid Factor Positive Arthritis, Non-ulcer Dyspepsia, Norwalk Viral Gastroenteritis, Obesity, Obsessive Compulsive Disorder, Pain Predominant FBD, Parkinson's Disease, Polyarteritis, Polyposis Coli, Primary Biliary Cirrhosis, Primary Clostridium difficile Infection (CDI), Primary Sclerosing Cholangitis (PSC), Pseudomembranous Colitis, Psychotic Disorders, Reiter's Syndrome, Relapsing Diverticulitis, Rett Syndrome, Rheumatoid Arthritis, Rosacea, Rotavirus Gastroenteritis, Sacroiliitis, Schizophrenia, Scleroderma, Sjogren's Syndome, Small Bowel Bacterial Overgrowth, Sudden Infant Death Syndrome (SIDS), Systemic Lupus Erythematosus, Ulcerative Colitis, Upper Abdominal FBD, Vasculitic Disorders, Viral Gastroenteritis, pre-diabetic syndrome, type I diabetes, type II diabetes, depression, schizophrenia, and a mood disorder.
Embodiment 100. A method for treating a disorder or condition in a subject in need thereof, comprising administering to said subject a therapeutically effective amount of a pharmaceutical composition of any one of embodiments 1 to 52 or a frozen fecal microbe composition of any one of embodiments 63 to 98, wherein said disorder or condition is selected from the group consisting of recurrent C. diff infection, autism spectrum disorder (ASD), constipation predominant functional bowel disease (FBD), pain predominant FBD, upper abdominal FBD, non-ulcer dyspepsia (NUD), gastro-oesophageal reflux, indeterminate colitis, microscopic colitis, pseudomembranous colitis, viral gastroenteritis, Norwalk viral gastroenteritis, rotavirus gastroenteritis, AIDS related gastroenteritis, non-rheumatoid factor positive arthritis, Lyme disease, systemic lupus, idiopathic thrombocytopenic purpura, Sjogren's syndrome, haemolytic uremic syndrome or scleroderma, Gillain-Barre syndrome, Chronic Inflammatory Demyelinating Polyneuropathy, chronic depression, schizophrenia, psychotic disorders, manic depressive illness, Asbergers syndrome, Rett syndrome, attention deficit hyperactivity disorder (ADHD), and attention deficit disorder (ADD), sudden infant death syndrome (SIDS), anorexia nervosa.
Embodiment 101. A method for treating a disorder or condition in a subject in need thereof, comprising administering to said subject a therapeutically effective amount of a pharmaceutical composition of any one of embodiments 1 to 52 or a frozen fecal microbe composition of any one of embodiments 63 to 98, wherein said disorder or condition is selected from the group consisting of recurrent or primary C. diff infection, autism spectrum disorder (ASD), ulcerative colitis, Crohn's disease, and irritable bowel syndrome.

While the present disclosure has been described with reference to particular embodiments, it will be understood by those skilled in the art that various changes may be made and equivalents may be substituted for elements thereof without departing from the scope of the present disclosure. In addition, many modifications may be made to adapt a particular situation or material to the teachings of the present disclosure without departing from the scope of the present disclosure.

Therefore, it is intended that the present disclosure not be limited to the particular embodiments disclosed as the best mode contemplated for carrying out the present disclosure, but that the present disclosure will include all embodiments falling within the scope and spirit of the appended claims.

### EXAMPLES

### Example 1: Testing of Various Lyoprotectants for Effective Preservation of Fecal Cell Viability

A study is designed to determine an effective lyoprotectant formulation for preserving the viability of microorganisms in lyophilized fecal microbiota preparations. Microbial cell membrane integrity is used as a surrogate marker for cell viability.

A liquid fecal microbiota extract is obtained via a process containing a homogenization step followed by a filtration step. In the homogenization step, a donated stool is mixed with a fixed ratio of homogenization buffer solution to stool to homogenize using a mechanical device to form stool slurry. After testing various ratios, an optimal ratio of 3 mL of homogenization buffer per gram of wet stool is established. The resulting stool slurry is filtered through a mesh filter (e.g., with a filter pore size of 330 μm) to generate a liquid fecal microbiota extract. This step removes a majority of the non-microbial fecal particulate matter present in the donor stool, including undigested food. Microbial cell viability is measured as in Example 2 based on cell membrane integrity. Membrane integrity of fresh donor material varies from 49% to 90%, even from the same donor. Equal or greater than 50% membrane integrity (% viability) is used to assess the quality of a liquid fecal microbiota extract.

The liquid fecal microbiota extract is then subject to further processing (e.g., lyophilization) to produce a lyophilized fecal microbiota preparation. Microbial cell viability is measured as in Example 2 both prior to and after the lyophilization step to determine a cell integrity maintenance ratio for each corresponding homogenization buffer. A cell integrity maintenance ratio is defined as the percent of microbial cells containing integral membrane post-lyophilization relative to pre-lyophilization.

Various homogenization buffer formulations are tested for their effect in maximizing microbial cell recovery from donor stool, as well as preserving membrane integrity (cell viability) during lyophilization. The present disclosure shows that 15% trehalose is found to be the most effective in maintaining cell membrane integrity and preserving cell viability during lyophilization. After a series of experiments, 15% trehalose are selected for their protective effect as measured by least impact on membrane integrity post freeze-thaw as well as post lyophilization. Representative data are presented below in Table 1 showing the effect of a homogenization buffer (0.25× Phosphate-buffered saline, pH 7.4 and 15% w/v of trehalose) over membrane integrity through a lyophilization process. For Donor #55, prior to lyophilization, 66.5% of microbes contain integral membrane. After lyophilization in the presence of 15% trehalose, 31.2% of microbes retain integral membrane, which amounts to a cell integrity maintenance ratio of about 46.9%. For Donor #62, prior to lyophilization, 70.9% of microbes contain integral membrane. After lyophilization in the presence of 15% trehalose, 43.7% of microbes retain integral membrane, which amounts to a cell integrity maintenance ratio of about 61.6%.

TABLE 1
Effect of Trehalose Concentration on Membrane Integrity

| Donor # | 55 | | | 62 | | |
|---|---|---|---|---|---|---|
| Membrane | 66.5% | | | 70.9% | | |
| Integrity | | | | | | |
| (% viability) | | | | | | |
| pre-lyophilization | | | | | | |
| Trehalose | 5% | 10% | 15% | 5% | 10% | 15% |
| concentration | | | | | | |
| Membrane | 4.4% | 11.6% | 31.2% | 15.2% | 25.3% | 43.7% |
| Integrity | | | | | | |
| (% viability) | | | | | | |
| post- | | | | | | |
| lyophilization | | | | | | |
| Cell integrity | 6.6% | 17.4% | 46.9% | 21.4% | 35.7% | 61.6% |
| maintenance | | | | | | |
| ratio | | | | | | |

### Example 2: Measurement of Microbial Cell Concentration and Cell Membrane Integrity

To monitor total cell number and determine proper dosage of a fecal microbiota preparation, cell concentration is measured using a fluorescence microscope-based method. Samples are serially diluted (from 1:10 to 1:10,000 folds) using sterile saline and then mixed with 1 μL of SYTO™ BC dye (Invitrogen, CA) to stain cells and 1 μL Prolong Anti-Fade Reagent (Invitrogen, CA) to minimize photo-bleaching of dye. The combined solution is mixed and incubated in the dark for 15 minutes at room temperature and analyzed using a Petroff-Hauser counting chamber.

Briefly, a 10 μl stained sample is applied to chamber, covered with a coverslip and visualized using an epifluorescence microscope (Nikon Corporation) equipped with a 40× objective, sola light engine and a filter specific for SYTO BC dye (Excitation 470 nm/Emission 525 nm).

The SYTO BC dye penetrates all intact microbial cells, whether alive or dead and these stained cells appear green when visualized in the epifluorescence microscope using the specific green filter (Excitation 470 nm/Emission 525 nm). Cells are counted in 5 squares. The process is repeated with 3 separate aliquots and average cells counted in each square are then used to calculate total cell concentration.

The membrane integrity (cell viability) is also measured using a fluorescence microscope-based method. The method is essentially the same as described above for cell concentration except two separate dyes are used. One aliquot is stained with SYTO BC as described above, and a second aliquot is stained with another dye, 1.5 μL Propidium Iodide (Invitrogen, CA).

Whereas the SYTO BC dye penetrates all cells which stain green, the Propidium Iodide (PI) dye only penetrates cells with a loss of membrane integrity (i.e. injured or dead cells), and the PI stained cells appear red when visualized under the epifluorescence microscope using the specific red filter (Excitation 560 nm/Emission 630 nm).

### Example 3: Cell Stability from Freeze and Thaw Cycles

The impact of repeated freeze-thaw cycles is evaluated over the cell stability of a fecal microbiota extract A liquid fecal microbiota extract from Example 1 is subject to repeated freeze-thaw (FT) cycles (FT1, FT2 and FT3) where one cycle is defined as freezing samples at −80° C. for a minimum of 24 hours followed by thawing at ambient temperature for a minimum of 30 minutes or until completely thawed as monitored visually. Results of these studies are provided below in Table 2. When mixed and extracted using a homogenization buffer (0.25× Phosphate-buffered saline, pH 7.4, 0.025% w/v L-Cysteine and 15% w/v of Trehalose), fecal material can be frozen and thawed at least two times without significantly impacting cell count and membrane integrity.

TABLE 2
Freeze-thaw stability of a fecal microbiota extract.

| | Cell Concentration (cells/mL) | | | | Membrane Integrity (% Viability) | | | |
|---|---|---|---|---|---|---|---|---|
| Donor | Initial (t0) | FT1 | FT2 | FT3 | Initial (t0) | FT1 | FT2 | FT3 |
| 42 | 7.3 × 10¹⁰ | 7.4 × 10¹⁰ | 8.4 × 10¹⁰ | 7.3 × 10¹⁰ | 65% | 55% | 50% | 36% |
| 55 | 8.4 × 10¹⁰ | 7.6 × 10¹⁰ | 7.2 × 10¹⁰ | 7.8 × 10¹¹ | 63% | 49% | 27% | 49% |
| 62 | 1.2 × 10¹¹ | 9.6 × 10¹⁰ | 1.1 × 10¹¹ | 8.6 × 10¹¹ | 69% | 53% | 47% | 36% |
| 63 | 3.1 × 10¹⁰ | 4.2 × 10¹⁰ | 3.4 × 10¹⁰ | 3.4 × 10¹⁰ | 64% | 49% | 42% | 38% |

### Example 4: Exemplary Clinical Treatment

A lyophilized fecal microbiota preparation described above comprising trehalose and Cysteine, is encapsulated in a delay-release capsule to open in the small intestine of patients. The capsules are stored at 2-8° C. Capsules are given to patients with microbiologically-proven second or fifth recurrent Clostridium difficile infection.

### Example 5: Testing of Various Reducing Aments for Effective Preservation of Fecal Cell Viability

A study is designed to determine an effective reducing agent formulation for preserving the viability of microorganisms in lyophilized fecal microbiota preparations. Microbial cell membrane integrity is used as a surrogate marker for cell viability.

Stool is collected from a donor and two samples are analyzed in parallel (0.05% Sodium Ascorbate, and 0.025% Cysteine) with stool divided from a single donor. The experiment is duplicated with stool from a second donor. A liquid fecal microbiota extract is obtained via a process containing a homogenization step followed by a filtration step as described in Example 1. In the homogenization step, a donated stool is mixed with a fixed ratio of homogenization buffer solution containing 0.05% w/v sodium ascorbate or 0.025% w/v cysteine.

The liquid fecal microbiota extract is then subject to further processing (e.g., lyophilization with 15% trehalose, blending, and encapsulation) to produce a lyophilized fecal microbiota preparation. Microbial cell viability is measured, as in Example 2, at the homogenization step, after the addition of trehalose (formulated drug substance step), after the lyophilization step, and after blending for each sample. Table 3 shows that both reducing agents, sodium ascorbate and cysteine, have higher membrane viability at each step.

TABLE 3
Effect of Reducing Agents on Membrane Integrity

| | Donor 1 | | | Donor 2 | | |
|---|---|---|---|---|---|---|
| | | 0.05% | | | 0.05% | |
| | | Sodium | 0.025% | | Sodium | 0.025% |
| Membrane Integrity | Control* | Ascorbate | Cysteine | Control* | Ascorbate | Cysteine |
| Homogenized stool | 70% | 49% | 42% | 61% | 65% | 54% |
| Formulated drug | 25% | 53% | 62% | 52% | 67% | 49% |
| Substance | | | | | | |
| Change | 45% | (4%) | (20%) | 9% | (2%) | 6% |
| Lyophilized Material | 36% | 35% | 41% | 48% | 41% | 39% |
| Encapsulation | 9.9% | 29% | 15% | 29% | 39% | 38% |
| Material | | | | | | |

*different donation
Percentages in parenthesis indicate a decrease.
Change indicates difference between homogenized stool and formulated drug substance.

A number of aspects have been described. Nevertheless, it will be understood that various modifications may be made without departing from the spirit and scope of the disclosure.

What is claimed is:

1. A pharmaceutical composition comprising a lyophilized fecal microbe preparation comprising a lyophilization formulation comprising at least 10% trehalose, wherein said lyophilization formulation is capable of providing a cell integrity maintenance ratio of at least 20%.

2. The pharmaceutical composition of claim 1, wherein said lyophilization formulation comprises at least 12.5%, at least 13%, at least 13.5%, at least 14%, at least 14.5%, at least 15%, at least 15.5%, at least 16%, at least 16.5%, at least 17%, at least 17.5%, at least 18%, at least 18.5%, at least 19%, at least 19.5%, at least 20%, at least 22.5%, at least 25%, at least 27.5%, at least 30%, at least 32.5%, at least 35%, at least 37.5%, at least 40%, at least 42.5%, at least 45%, at least 47.5%, at least 50%, at least 52.5%, at least 55%, at least 57.5%, or at least 60% trehalose.

3. The pharmaceutical composition of claim 1 or 2, wherein said lyophilization formulation is capable of providing a cell integrity maintenance ratio of at least 22.5%, at least 25%, at least 27.5%, at least 30%, at least 32.5%, at least 35%, at least 37.5%, at least 40%, at least 42.5%, at least 45%, at least 47.5%, at least 50%, at least 52.5%, at least 55%, at least 57.5%, at least 60%, at least 62.5%, at least 65%, at least 67.5%, at least 70%, at least 72.5%, at least 75%, at least 77.5%, at least 80%, at least 82.5%, at least 85%, at least 87.5%, at least 90%, at least 92.5%, or at least 95%.

4. The pharmaceutical composition of any one of claims 1 to 3, wherein said lyophilization formulation further comprises a reducing agent.

5. The pharmaceutical composition of claim 4, wherein said reducing agent is cysteine selected from the group consisting of D-cysteine and L-cysteine, or sodium abscorbate.

6. The pharmaceutical composition of claim 5, wherein said reducing agent comprises about 0.025% cysteine.

7. The pharmaceutical composition of any one of the preceding claims, wherein said fecal microbe preparation comprises a non-selected and substantially complete fecal microbiota preparation from a single donor.

8. The pharmaceutical composition of claim 7, wherein the preparation of said fecal microbe preparation involves a treatment selected from the group consisting of ethanol treatment, detergent treatment, heat treatment, irradiation, homogenization, sonication, and combination thereof.

9. The pharmaceutical composition of claim 7, wherein the preparation of said fecal microbe preparation involves a separation step selected from the group consisting of filtering, sieving, density gradients, filtration, chromatography, sedimentation, centrifugation, and a combination thereof.

10. The pharmaceutical composition of claim 7, wherein said lyophilized formulation further comprises one or more cryoprotectants selected from the group consisting of dimethyl sulfoxide (DMSO), glycerol, polyethylene glycol (PEG), alanine, glycine, proline, mannitol, sucrose, glucose, lactose, ribose, hydroxypropyl-β-cyclodextrin (HPβCD), and any combination thereof.

11. The pharmaceutical composition of claim 7, wherein said pharmaceutical composition is for oral administration.

12. The pharmaceutical composition of claim 7, wherein said pharmaceutical composition is formulated as a geltab, pill, microcapsule, capsule, or tablet.

13. The pharmaceutical composition of claim 7, wherein every 200 mg of said pharmaceutical composition comprises a pharmacologically active dose of microbes or spores selected from the group consisting of 10³ to 10¹⁵, 10⁴ to 10¹⁵, 10⁵ to 10¹⁵, 10⁶ to 10¹⁵, 10⁷ to 10¹⁵, 10⁸ to 10¹⁵, 10³ to 10¹⁴, 10⁴ to 10¹⁴, 10⁵ to 10¹⁴, 10⁶ to 10¹⁴, 10⁷ to 10¹⁴, 10⁸ to 10¹⁴, 10⁴ to 10¹³, 10⁵ to 10¹², 10⁶ to 10¹¹, 10⁷ to 10¹⁰, 10⁸ to 10⁹, 10³ to 10¹³, 10³ to 10¹², 10³ to 10¹¹, 10³ to 10¹⁰, 10³ to 10⁹, 10³ to 108, 10³ to 10⁷, 10³ to 10⁶, 10³ to 10⁶, and 10³ to 10⁴ cfu or total cell count.

14. The pharmaceutical composition of claim 7, wherein said fecal microbe preparation has at least about 10%, 20%, 30%, 40%, 50%, 60%, 70%, 80%, 85%, 90%, 95%, 99%, or 99.5% microbes in a spore form.

15. The pharmaceutical composition of claim 7, wherein said pharmaceutical composition is effective for treating one or more disorders selected from the group consisting of recurrent or primary C. diff infection, autism spectrum disorder (ASD), ulcerative colitis, Crohn's disease, and irritable bowel syndrome.

16. A method of preparing a lyophilized fecal microbe preparation, said method comprising:
a. obtaining a liquid fecal microbe preparation in a lyophilization formulation comprising between 10% and 20% trehalose; and
b. freeze-drying said liquid fecal microbe preparation, wherein said lyophilization formulation is capable of providing a cell integrity maintenance ratio of at least about 30%.

17. The method of claim 16, wherein said lyophilization formulation further comprises L-cysteine.

18. The method of claim 16 or 17, wherein said liquid fecal microbe preparation is obtained from fecal material without culturing fecal microbes in said fecal material.

19. A frozen fecal microbe composition comprising trehalose, wherein said frozen fecal microbe composition is capable of being thawed at least once without loss of microbial cell membrane integrity of more than 50%.

20. The frozen fecal microbe composition of claim 19, wherein said composition further comprises cysteine selected from the group consisting of D-cysteine and L-cysteine.

21. The frozen fecal microbe composition of any one of claims 19 to 20, wherein said fecal microbe composition comprises a non-selected and substantially complete fecal microbiota preparation from a single donor.

22. The frozen fecal microbe composition of claim 19, wherein said composition further comprises one or more cryoprotectants selected from the group consisting of dimethyl sulfoxide (DMSO), glycerol, polyethylene glycol (PEG), alanine, glycine, proline, mannitol, sucrose, glucose, lactose, ribose, hydroxypropyl-β-cyclodextrin (HPβCD), and any combination thereof.

23. The frozen fecal microbe composition of claim 19, wherein said composition is for oral administration.

24. The frozen fecal microbe composition of claim 19, wherein every 200 mg of said composition comprises a pharmacologically active dose of microbes or spores selected from the group consisting of 10³ to 10¹⁴, 10⁴ to 10¹⁴, 10⁵ to 10¹⁴, 10⁶ to 10¹⁴, 10⁷ to 10¹⁴, 10⁸ to 10¹⁴, 10⁴ to 10¹³, 10⁵ to 10¹², 10⁶ to 10¹¹, 10⁷ to 10¹⁰, 10⁸ to 10⁹, 10³ to 10¹³, 10³ to 10¹², 10³ to 10¹¹, 10³ to 10¹⁰, 10³ to 10⁹, 10³ to 10⁸, 10³ to 10⁷, 10³ to 10⁶, 10³ to 10⁵, and 10³ to 10⁴ cfu or total cell count.

25. The frozen fecal microbe composition of claim 19, wherein said fecal microbe composition has at least about 20%, 30%, 40%, 50%, 60%, 70%, 80%, 85%, 90%, 95%, 99%, or 99.5% microbes in a spore form.

26. A method for treating a disorder or condition in a subject in need thereof, comprising administering to said subject a therapeutically effective amount of a pharmaceutical composition of any one of claims 1 to 15 or a frozen fecal microbe composition of any one of claims 19 to 25, wherein said disorder or condition is selected from the group consisting of recurrent or primary C. diff. infection, autism spectrum disorder (ASD), ulcerative colitis, Crohn's disease, irritable bowel syndrome, constipation predominant functional bowel disease (FBD), pain predominant FBD, upper abdominal FBD, non-ulcer dyspepsia (NUD), gastro-oesophageal reflux, indeterminate colitis, microscopic colitis, pseudomembranous colitis, viral gastroenteritis, Norwalk viral gastroenteritis, rotavirus gastroenteritis, AIDS related gastroenteritis, non-rheumatoid factor positive arthritis, Lyme disease, systemic lupus, idiopathic thrombocytopenic purpura, Sjogren's syndrome, haemolytic uremic syndrome or scleroderma, Gillain-Barre syndrome, Chronic Inflammatory Demyelinating Polyneuropathy, chronic depression, schizophrenia, psychotic disorders, manic depressive illness, Asbergers syndrome, Rett syndrome, attention deficit hyperactivity disorder (ADHD), and attention deficit disorder (ADD), sudden infant death syndrome (SIDS), anorexia nervosa.
